(19) United States
(12) Patent Application Publication
(10) Pub. No.: US 2020/0326333 A1
(43) Pub. Date: Oct. 15, 2020

(54) DIAGNOSIS AND TREATMENT OF AUTOIMMUNE DISEASES

(71) Applicant: OP-T LLC, Denver, CO (US)

(72) Inventors: David Hal Wagner, Jr., Denver, CO (US); Martin Glenn Yussman, Denver, CO (US); Charles W. Henry, Denver, CO (US); Gisela M. Vaitaitis, Centennial, CO (US)

(73) Assignee: OP-T LLC, Denver, CO (US)

(21) Appl. No.: 16/846,107

(22) Filed: Apr. 10, 2020

Related U.S. Application Data
(60) Provisional application No. 62/833,593, filed on Apr. 12, 2019.

(51) Int. Cl. G01N 33/532; G01N 15/14

(52) U.S. Cl. CPC G01N 33/532; G01N 2015/0065; G01N 2800/042; G01N 15/14

(57) ABSTRACT

Methods and cellular markers useful for methods of diagnosing autoimmune disease, including type 1 diabetes (T1D) and disease progression in T1D patients, as well as identifying treatments for and monitoring treatment of patients with T1D. Methods of treating patients having T1D.

Some aspects of the developments herein were made with government support under grant numbers 1R41AI131784-01, 1R42DK115296-01, and 4R42DK115296-02 awarded by the National Institutes of Health. The government may have certain rights in the developments hereof.

### FIELD

The present developments relate to the identification and use of cellular markers useful in the diagnosis of Diabetes Mellitus Type 1 (also referred to as Type 1 Diabetes or T1D), changes in disease states and disease progression in T1D patients. These markers are also useful for identifying treatments for and monitoring treatment of patients with Type 1 Diabetes and other autoimmune diseases.

### BACKGROUND

Type 1 Diabetes (T1D) is an autoimmune disease that affects the β-islets of the pancreas, leading to the loss of insulin production and ultimately to overt disease signified by hyperglycemia. While T1D has clear genetic components, the incidence of T1D is increasing much more than genetics would predict (1), and the overall incidence of diabetes in the world is on the rise. T1D accounts for 5-10% of all cases of diabetes and it is estimated that more than 150,000 individuals, worldwide, are diagnosed with the disease annually (2). Recent work proposes that human T1D is a relapsing-remitting disease with immunologic and metabolic factors affecting the different phases of progression towards disease as well as the honeymoon phase, during which the pancreas recovers some of the ability to produce insulin (3, 4). Because the disease is autoimmune, its affects are cumulative. T cells and other immune cells infiltrate the pancreatic islets over time, in some cases extended time-periods, and because of this, various stages of a prediabetic state exist. In human subjects, there is no hypothesis for why some individuals onset at less than 5 years of age and others onset as late as 45 or even 50 years of age. Determining what the prediabetic stages represent during diabetogenesis may in some instances affect treatment options.

Genetic factors, such as the HLA DR3 and DR4 as well as DQ2 and DQ8 haplotypes, are associated with increased risk of developing T1D, but other environmental factors also play a role. It was shown that, in T1D, proinsulin peptides form hybrids with other peptides through covalent cross-linking, forming neo-antigens that can be recognized by pathogenic CD4 T cells (5-7). It is likely that factors that are affected by both the environment and genetics contribute to the risk of developing disease (8). One such factor would be epitope spreading (3) which involves the ability of T cells to alter their T cell receptors (TCR) such that they can recognize new β-cell autoantigens (8). That process would be influenced by the availability of neo antigens and immune stimulation (environment) as well as presentation of those new antigens by HLA molecules (genetic) (8). No single factor alone is able to predict who will develop T1D, therefore it is desirable to identify robust methods to predict who is at high risk of developing T1D. Such methods may include combinations of genetic markers, environmental factors and immune system inflammatory markers.

A unique T cell subset has been shown to be involved in the development of autoimmune disease. These cells are phenotypically characterized as CD4+ and CD40+ (Waid, D. M., et al., Eur. J of Immunol., 34:1488, 2004; Vaitaitis, G. M., et al., Cutting Edge, J. Immunol., 170:3455, 2003; Wagner, D. H., Jr., et al., Proc. Nat'l. Acad. Sci. USA, 99:3782, 2002; Wagner, D. H., Jr., et al., Int'l J. of Mol. Med. 4:231, 1999), and are referred to as Th40 cells. (Waid, D. M., et al. Eur. J. of Immunol. 34:1488, 2004; Vaitaitis, G. M., et al., Cutting Edge, J. Immunol. 170:3455, 2003; Wagner, D. H., Jr., et al., Proc. Nat'l Acad. Sci. USA 99:3782, 2002; Wagner, D. H., Jr., et al., Int'l J. of Mol. Med. 4:231, 1999). CD40 expression typically is associated with antigen presenting cells and the majority of prior developments describes CD40 as being expressed on B cells, macrophages, monocytes, and other cells; however, CD40 proteins are also expressed on T cells (Waid, D. M., et al., 2004. Eur. J of Immunol., 34:1488, 2004; Vaitaitis, G. M., et al., Cutting Edge, J. Immunol., 170:3455, 2003; Wagner, D. H., Jr., et al., Proc. Nat'l Acad. Sci. USA, 99:3782, 2002; Wagner, D. H., et al., Int'l. J. of Mol. Med., 4:231, 1999; Bourgeois, C., et al., Science, 297:2060, 2002; Fanslow, W. C., et al., J. of Immun., 152:4262, 1994; Ramsdell, F., et al., J. of Immunol. 152:2190, 1994; Grabstein, K. H., et al., J. of Immunol., 150:3141, 1993; Armitage, R. J., et al., Sem. in Immun., 5:401, 1993; Cooper, C. J., et al., J. of Immunol., 173:6532, 2004). While Th40 cells include a proportion of the peripheral CD4+ compartment in näive, non-autoimmune mice (Waid, D. M., et al., Eur. J. of Immunol., 34:1488, 2004; Wagner, D. H., Jr., et al., Int'l J. of Mol. Med., 4:231, 1999; Waid, D. M., et al., J. of Neuroimmunol., 270:1-2:75, 2014), and in humans (Waid. D. M., et al., Clin. Immunol., 124:138, 2007), this proportion is drastically expanded to as much as 50% of the CD4+ compartment in autoimmune prone mice (Waid, D. M., et al., Eur. J. of Immunol. 34:1488, 2004; Wagner, D. H., Jr., et al., Proc. Nat'l Acad. Sci. USA 99:3782, 2002; Wagner, D. H., et al., Int'l J. of Mol. Med., 4:231, 1999) and humans (Waid. D. M., et al., Clin. Immunol. 124:138, 2007; Waid, D. M., et al., J. of Neuroimmunol., 270:1-2:75, 2014). These T cells do not express early activation markers and occur in the naïve phenotype of non-challenged mice.

In NOD (non-obese diabetic) mice, a mouse model of Type 1 Diabetes, Th40 cells occur at exaggerated levels in spleen, lymph nodes and the pancreas, even prior to diabetes onset (Waid, D. M., et al., Eur. J. of Immunol. 34:1488, 2004; Wagner, D. H., Jr., et al., Proc. Nat'l Acad. Sci. USA 99:3782, 2002). An elevated number and percentage of these T cells are seen in peripheral blood of type 1 diabetic (T1D) patients when compared to non-autoimmune controls and type 2 diabetic patients (Waid. D. M., et al., Clin. Immunol., 124:138, 2007).

The observed increase in Th40 cells could mean that those T cells are antigen responsive or that CD40 expression is activation induced. Furthermore, several diabetogenic T cell clones are CD40+ (Wagner, D. H., Jr., et al., Proc. Nat'l Acad. Sci. USA 99:3782, 2002). Purified primary Th40 cells from NOD mice and from pre-diabetic NOD (12-weeks of age) mice successfully transfer type 1 diabetes to NOD/scid (Non-Obese Diabetic/Severe Combined Immunodeficiency) recipient mice, directly demonstrating pathogenicity of the Th40 T cell subset (Waid, D. M., et al., Eur. J. of Immunol. 34:1488, 2004; Wagner, D. H., Jr., et al., Proc. Nat'l Acad. Sci. USA, 99:3782, 2002). It has been shown that Th40 cells infiltrate islet beta cells destroying insulin production thus suggesting islet antigen specificity (Waid, D. M., et al., Eur. J. of Immunol. 34:1488, 2004; Wagner, D. H., Jr., et al., Proc. Nat'l Acad. Sci. USA 99:3782, 2002). It has also been shown that Th40 cells are required for diabetes transfer. Peripheral (spleen and regional lymph node) T cells that were CD40 depleted, then CD25, Treg, depleted were not capable of transferring diabetes to Scid (Severe Combined Immunodeficiency) recipients. Even though Treg cells were removed, if the auto-aggressive CD40+ T cells subset is absent, disease transfer does not occur.

While Th40 cells are important in the development of autoimmunity, another important factor is expression of the CD40-Ligand, CD154. CD154 is temporally induced on activated T-cells in response to CD3/TCR stimulation (Lederman, S. et al., J. of Exp. Med., 175:1091, 1992). CD154 expression has also been demonstrated on platelets, monocytes, basophils, eosinophils, dendritic cells, fibroblasts, smooth muscle, and endothelial cells (Russo, S. et al., J. Immunol. 171:5489, 2003; Stumpf, C., et al., Eur. J. Heart Fail., 5:629, 2003; Schonbeck, U., et al., Cell Mol. Life Sci. 58:4, 2001). CD154 is a member of the tumor necrosis factor (TNF) super-family and a soluble form of CD154 (sCD154) has been described. (Russo, S., et al., J. Immunol. 171:5489 2003; Stumpf, C., et al., Eur. J. Heart Fail 5:629, 2003; Toubi, E., et al., Autoimmunity 37:457, 2004). Therefore, sCD154 may act like a cytokine. (Stumpf, C., et al., Eur. J. Heart Fail. 5:629, 2003). Even though CD154 has not been genetically linked in T1D studies, sCD154 is significantly elevated in T1D and may play a role in the disease process (Varo, N. et al., Circulation 107:2664, 2003; Cipollone, F., et al., Diabetologia 48:1216, 2005; Devaraj, S., et al., Diabetes 55:774, 2006). The importance of CD40-CD154 interaction in autoimmunity has been established (Wagner, D. H., Jr., et al., Proc. Nat'l Acad. Sci. USA 99:3782, 2002; Kobata, T., et al., Rev. Immunogenet. 2:74, 2000; Homann, D., et al., Immunity 16:403, 2002; Goodnow, C. C., et al., Lancet 357:2115, 2001; Balasa, B., et al., J. of Immunol. 159:4620, 1997). Blocking CD40-CD154 interaction may prevent collagen induced arthritis, (Durie, F. H., et al., Science 281:1328, 1993) experimental autoimmune encephalitis (Howard, L. M., et al., Autoimmunity 37:411, 2004), prostatitis (Grossman, M. E., et al., J. Immunother. 24:237, 2001), and T1D in the NOD mouse model (Durie, F. H. et al., Science 281:1328, 1993; Balasa, B. et al., Journal of Immunology 159:4620, 1997; Howard, L. M., et al., Autoimmunity 37:411, 2004; Grossman, M. E. et al., J. Immunother. 24:237, 2001). In the diabetes model, benefits were found in administering a CD154 blocking antibody to NOD mice at 3-weeks of age because at 9-weeks, blocking antibodies had no effect on diabetes prevention (Balasa, B. et al., J. of Immunol. 159:4620, 1997).

CD40 is a 50-kDa integral membrane protein of the tumor necrosis factor receptor (TNF-R) family. It is constitutively expressed as a homotrimer (Foy T M, et al., Ann. Rev. of Immunol., 14:591, 1996). In general, stimulation of all CD40-expressing cell types induces operations which contribute to inflammation, such as enhancement of costimulatory and adhesion molecules, and up-regulation of proteolytic enzymes (Mach, F. et al., Atherosclerosis. 137 Suppl:S89-95, 1998).

CD40's ligand—CD154—is a 39-kDa protein that belongs to the tumor necrosis factor (TNF) family. CD40 forms a trimer that binds CD154 at the interface of the three monomers. CD154 is expressed commonly on cells beyond the surface-expressed CD154, as CD154 may also exist in a soluble biologically active form (sCD154) that is shed from the cell surface after activation. The main source of sCD154 is platelets. (Kaufman, J., et al., J. of Thrombosis and Haemostasis, 5:788-96, 2007).

CD154 is known to interact with αMβ2 (CD11b/CD18), α5β1 (CD49e/CD29), and αIIb β3 (CD41/CD61) found on platelets. Moreover, CD154 has been found to interact with several other integrins including αL (CD11a), αM (CD11b), αD (CD11d), β4 (CD104), α3 (CD49c), and α5 (CD49e).

Previously, inventors hereof identified a CD4 T cell subset, characterized by the expression of CD40 (Th40 cells), which is significantly (p<0.0001) expanded in number in peripheral blood from T1D patients but not from non-autoimmune control or non-autoimmune T2D patients (9). That expansion was present in T1D patients regardless of HLA haplotype and, in a blinded study, we successfully identified T1D patients versus control subjects with 95% accuracy. Similar to our findings in human T1D patients, we found an expansion of Th40 cells in the non-obese diabetic (NOD) mouse model of T1D (10-14). Unlike humans, T1D in NOD mice runs a consistent, predictable course. In those mice, Th40 cells progressively expand in the prediabetic stages (3-12 weeks of age), concurrently with establishment of insulitis (10-14). In very young NOD mice, in the pre-insulitis stage, peripheral Th40 cells are at numbers equivalent to controls, but expansions already occur in pancreatic lymph nodes (15). Eventually overt hyperglycemia ensues, at which stage peripheral Th40 cell numbers reach up to 60% compared to about 10-15% in control mice (10,12). Th40 cells are also necessary and sufficient to transfer T1D from both prediabetic (with present insulitis) and diabetic NOD mice to NOD.scid mice (10,12,14). Interestingly, Th40 cells are capable of reactivating recombination activating gene (RAG) 1 and 2 which leads to alteration of the TCRs expressed by these mature, peripheral T cells (16,17). Such alterations could potentially lead to epitope spreading, thus expanding the TCR repertoire.

Given that Th40 cells are readily detected in peripheral blood, that early detection and prediction may have an affect on treatment, and the difficulty of obtaining a proper diagnosis, more effective methods for detection, prediction, diagnosis, and treatment may be desired. CD40 is an apparent player in several autoimmune diseases including, diabetes, arthritis, colitis, EAE (the mouse model for MS) (Girvin et al., 2002) and in human MS (Benveniste, et al., 2004; Giuliani et al., 2005). CD40 as a dominant player in so many diverse autoimmune diseases may suggest that it constitutes an interesting and early phase autoimmune inflammation marker. Thus, reliable and effective methods for identifying, treating, and/or preventing autoimmune diseases may be desired. The present developments may address these desires.

### SUMMARY

The present developments provide, inter alia, a method to diagnose a subject as having type 1 diabetes, which in some implementations may include: determining the percentage, count, and/or level of Th40 cells in a sample isolated from the subject, comparing the percentage, count, and/or level of Th40 cells to a control sample or a standard value, diagnosing type 1 diabetes in the subject, an increase in the percentage, count, and/or level of Th40 cells in the sample from the subject relative to the control sample or standard may be indicative of type 1 diabetes in the subject.

The developments hereof may further provide a method to diagnose relapse or disease progression in a subject having type 1 diabetes that is being treated for type 1 diabetes ins some instances including: determining the percentage, count, and/or level of Th40 cells in a sample isolated from the subject, comparing the percentage, count, and/or level of Th40 cells in the sample from the subject to a control sample or a standard value, and diagnosing relapse or disease progression, the percentage, count, and/or level of Th40 cells in the sample from the subject indicating the subject is in relapse or has disease progression.

The developments may further provide a method to identify a composition useful for the treatment of type 1 diabetes in a subject including: administering a test composition to the subject, evaluating whether the administration of the test composition causes a transient change in the percentage, count, and/or level of Th40 cells in a sample isolated from the subject, a transient decrease in the percentage, count, and/or level of Th40 cells in samples from the subject over about 1-6 days perhaps indicating the test composition may be useful for the treatment of type 1 diabetes.

The developments hereof may also provide a method to identify a dose of a composition, useful for the treatment of type 1 diabetes in a subject, to elicit a desired magnitude of response, including: administering the composition to the type 1 diabetes subject at different doses, evaluating the change in the percentage, count, and/or level of the Th40 cells in a sample isolated from the type 1 subjects at different doses of the composition; and identifying a dose of the composition that elicits the desired magnitude of decrease in the percentage, count, and/or level of Th40 cells in samples from the subject over about 1-6 days indicating the test composition may be useful for treatment of type 1 diabetes.

The developments hereof may further provide a method to monitor the treatment of type 1 diabetes in a subject, including: administering a therapeutic composition to the subject, and evaluating whether administration of the therapeutic composition causes a transient decrease in the percentage, count, and/or level of Th40 cells in a sample isolated from the type 1 diabetes subject following the administration of the composition, a transient decrease in the percentage, count, and/or level of Th40 cells in the samples from the subject perhaps indicating efficacious treatment of type 1 diabetes in the subject.

In some implementations of these methods, the control sample is a sample from at least one subject known not to have type 1 diabetes. In some aspects of these methods, the control sample is a sample from at least one subject known to have type 1 diabetes. In some aspects of these methods, the control sample is a sample obtained from the subject at an earlier date. In some aspects of these methods, the control sample is a sample obtained from at least one subject known to have a disease selected from the group of multiple sclerosis, relapsing-remitting multiple sclerosis (RRMS), primary progressive multiple sclerosis (PPMS), secondary-progressive multiple sclerosis (SPMS), Clinically isolated syndrome (CIS), and Radiologically isolate syndrome (RIS). In some implementations of these methods, the control sample is a sample obtained from at least one Type-2 Diabetes (T2D) patient. In some aspects of these methods, the control sample is a sample from at least one subject who does not have an autoimmune disease.

The developments hereof provide a method to diagnose a subject as having type 1 diabetes, including: determining the percentage of cells expressing CD40, determining the percentage of cells expressing one or more markers selected from CD4, CD8, CD40, CD25, CD45, TCRV8.3+, CXCR3 and CCR5, comparing the percentage of Th40 cells to a control sample or a standard value, comparing the percentage of CD4, CD8, CD40, CD25, CD45, TCRV8.3+, CXCR3 and CCR5 cells to a control sample or a standard value, diagnosing type 1 diabetes in the subject wherein an increase in the percentage of Th40 and an increase or decrease in the percentage of CD4, CD8, CD40, CD25, CD45, TCRV8.3+, CXCR3 and CCR5 cells in the sample from the subject relative to the control sample or standard is indicative of type 1 diabetes in the subject.

The developments hereof provide a method to diagnose type 1 diabetes in a subject including: determining the percentage of cells expressing CD11a, CD11b, CD11d, CD18, CD29, CD41, CD49c, CD49e, CD61, and CD104 from the subject, comparing the percentage of CD11a, CD11b, CD11d, CD18, CD29, CD41, CD49c, CD49e, CD61, and CD104 cells to a control sample or a standard value, diagnosing type 1 diabetes in the subject wherein an increase or decrease in the percentage of CD11a, CD11b, CD11d, CD18, CD29, CD41, CD49c, CD49e, CD61, and CD104 cells in the sample from the subject relative to the control sample or standard is indicative of type 1 diabetes in the subject.

The developments hereof may also provide a method to identify and diagnose autoimmune disease in a subject including: determining the percentage of cells expressing one or more markers selected from CD4, CD8, CD40, CD25, CD45, TCRV8.3+, CXCR3, CCR5, CD11a, CD11b, CD11d, CD18, CD29, CD41, CD49c, CD49e, CD61, and CD104, comparing the percentage of one or more markers selected from CD4, CD8, CD40, CD25, CD45, TCRV8.3+, CXCR3, CCR5, CD11a, CD11b, CD11d, CD18, CD29, CD41, CD49c, CD49e, CD61, and CD104 to a control sample or a standard value, determining the one or more markers selected from CD4, CD8, CD40, CD25, CD45, TCRV8.3+, CXCR3, CCR5, CD11a, CD11b, CD11d, CD18, CD29, CD41, CD49c, CD49e, CD61, and CD104 that are outside of a standard range of values; and diagnosing a disease in the subject selected from the group including: type 1 diabetes (T1D), multiple sclerosis, systemic lupus erythematosus, rheumatoid arthritis, Crohn's disease, inflammatory bowel disease, chronic obstructive pulmonary disease, autoimmune asthma, atherosclerosis, vasculitis, hypertension, thyroiditis, Hashimoto's disease, Graves disease, primary biliary cirrhosis, Paget's disease, Addison's disease, acute respiratory distress syndrome, acute lung injury, and amyotrophic lateral sclerosis.

The developments hereof may also include one or more implementations that include a determination of levels of autoantibodies to β-cell associated antigens and HLA haplotypes.

The developments hereof may also include the staining or measurement of cell markers an in some instances may use antibodies including: anti-CD40 (G28-5; conjugated to Alexa-Fluor 405); anti-CD3 (UCHT1; PerpCp-cy5.5); anti-cd4 (RPA-T4; APC-Cy7); anti-CD8 (OKT8; APC); anti-CD45RO (UCHL1; PE); anti-IL-2 (MQ1-18H12; PE); anti-IL-4 (8D4-8; PE-CY7); anti-IL-6 (MQ2-13A5; PE), anti-IL-10 (JE53-9D7; PE), anti-IL-17α (eBio64DEC17; APC), anti-IFNγ (45.B3; FITC) and anti-TNFα (Mab11; PE).

These as well as other alternative and/or additional aspects are exemplified in a number of illustrated alternative and/or additional implementations and applications, some of which are shown in the figures and characterized in the claims section that follows. However, as will be understood by the ordinarily skilled artisan, the above summary and the detailed description below do not describe the entire scope of the developments hereof and are indeed not intended to describe each illustrated embodiment or every implementation of the present developments nor provide any limitation on the claims or scope of protection herein set forth below.

### BRIEF DESCRIPTION OF THE DRAWINGS

The drawings include:

FIG. 1A and FIG. 1B provide graphs of relationships found between control and T1D subjects.

FIG. 2A and FIG. 2B provide graphs of conventional CD4 T cells from stratified pre-T1D subjects.

FIG. 3A-1, FIG. 3A-2, FIG. 3B, and FIG. 3C provide graphs and charts of stratification of preT1D subjects and cytokine profiles.

FIG. 4A, FIG. 4B, FIG. 4C, FIG. 4D, FIG. 4E-1, and FIG. 4E-2 provides dot plots, histograms, and charts of differences in presence of biomarkers.

FIG. 5A, FIG. 5B-1, and FIG. 5B-2 provide graphs of Th40 cell levels in subjects.

FIG. 6A and FIG. 6B provide graphs of Th40 levels in subjects.

FIG. 7A, FIG. 7B and FIG. 7C provides graphs of different Th40 cell levels from subjects.

FIG. 8A, FIG. 8B, FIG. 8C, FIG. 8D, FIG. 8E, FIG. 8F, FIG. 8G, and FIG. 8H provide graphs and charts related to haplotypes in subjects.

FIGS. 9A and 9B provide tables related to the subjects described in examples hereof.

FIG. 10A, FIG. 10B, FIG. 10C-1, FIG. 10C-2, FIG. 10D, FIG. 10E, FIG. 10F, FIG. 10G, FIG. 10H-1, FIG. 10H-2, FIG. 10I, FIG. 10J-1, FIG. 10J-2, and FIG. 10J-3 provide blots, tables, graphs, charts, and plots related to additional biomarkers, receptors, and peptides that may bind thereto.

### DETAILED DESCRIPTION

The present developments include one or more of methods to diagnose a subject as having type 1 diabetes, methods to diagnose disease progression or relapse in a subject having type 1 diabetes, methods to identify a composition useful for the treatment of type 1 diabetes, methods to identify an appropriate dose of a composition useful for the treatment, prevention, or reversal of type 1 diabetes, and/or methods to monitor treatment of a subject for type 1 diabetes.

Before the present developments are further described, it is to be understood that these developments are not strictly limited to particular implementations described, as such may of course vary. It is also to be understood that the terminology used herein is for the purpose of describing particular implementations only, and is not intended to be limiting, since the scope of the present developments will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably.

Unless defined otherwise, all technical and scientific terms used herein have the same meaning as commonly understood by one of ordinary skill in the art to which these developments belong. Although any methods and materials similar or equivalent to those described herein can also be used in the practice or testing of the present developments, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present developments are not entitled to antedate such publication by virtue of prior invention. Further, the dates of publication provided may be different from the actual publication dates, which may need to be independently confirmed.

It is appreciated that certain features of the developments, which are, for clarity, described in the context of separate implementations, may also be provided in combination in a single implementation. Conversely, various features of the developments, which are, for brevity, described in the context of a single embodiment, may also be provided separately or in any suitable sub-combination. All combinations of the implementations are specifically embraced by the present developments and are disclosed herein just as if each and every combination was individually and explicitly disclosed. In addition, all sub-combinations are also specifically embraced by the present developments and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Furthermore, as used herein the term animal refers to a vertebrate, preferably a mammal, more preferably a human. Suitable mammals on which to use the methods of the present developments include but are not limited farm animals, sports animals, pets, primates, mice, rats, horses, dogs, cats, and humans. The term animal can be used interchangeably with the terms subject or patient.

As used herein, the terms “patient”, “a subject who has T1D”, “a patient who has T1D”, “a T1D subject”, “a T1D patient”, and similar phrases, are intended to refer to subjects who have been diagnosed with type 1 diabetes (T1D) or pre-Type 1 Diabetes (pre-T1D). The terms “Healthy subject”, “non-T1D subject”, “a subject who does not have T1D”, “a patient who does not have T1D”, and similar phrases, are intended to refer to a subject who has not been diagnosed with T1D. A Healthy subject has no other acute systemic disease.

As used herein, the term “sample” or “biological sample” includes a sample of any celltype or from any tissue or body fluid, body fluids including, but not limited to: cerebrospinalfluid (CSF), serum, plasma, blood, or fluid from any suitable tissue. In a preferred implementation, the biological sample is blood or any component of blood (e.g., serum, plasma, etc.).

Candidate test compositions identified or designed by the methods of the developments can be synthesized using techniques known in the art, and depending on the type of compound. Synthesis techniques for the production of non-protein compounds, including organic and inorganic compounds are well known in the art. For example, for smaller peptides, chemical synthesis methods may be preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas IO Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TF A/scavenger cleavage mixture. A compound that is a protein or peptide can also be produced using recombinant DNA technology and methods standard in the art, particularly if larger quantities of a protein are desired.

As used herein, the terms “test composition”, “test compound”, “putative inhibitory compound” or “putative regulatory compound” refer to compositions having an unknown or previously unappreciated regulatory activity in a particular process. As such, the term “identify” with regard to methods to identify compounds is intended to include all compositions, the usefulness of which as a regulatory compound for the purposes of regulating the expression or activity of a target biomarker or otherwise regulating some activity that may be useful in the study or treatment of T1D is determined by a method of the present developments.

The terms “cellular marker”, “biomarker” or “marker”, as used herein, can refer to a cell, particularly a blood cell, a ratio of particular cells, a cell-associated polypeptide or protein, a soluble polypeptide or metabolite described herein or to a polynucleotide (including a gene) that encodes a polypeptide identified by the developments. In addition, the terms “biomarker” or “marker” can be generally used to refer to any portion or component of such a cell, portion or component indicating the ratio of particular cells, cell-associated polypeptides, soluble polypeptides or polynucleotides that can identify or correlate with the cell, ratio of particular cells, full-length polypeptide or polynucleotide, for example, in an assay of the developments hereof.

Biomarkers also include any components or portions of cells, precursors and successors of polypeptides and polynucleotides of the developments, as well as polypeptides and polynucleotides substantially homologous to polypeptides and polynucleotides of the developments. Accordingly, a biomarker useful in the present developments is any cell, cell ratio, polynucleotide, polypeptide or metabolite, the expression or occurrence of which is regulated (up or down) in a subject with a condition (e.g., T1D) as compared to a normal control.

Of the cells analyzed, the present developments identify multiple biomarkers, (i) the expression of which are regulated differentially in subjects with T1D as compared to subjects without T1D, (ii) that cause short-term changes upon drug therapy, and (iii) that cause longer term changes upon drug therapy among the different cohort populations.

The present developments include the use of any of the biomarkers as described herein (including genes or their RNA or protein products), as targets for the development or identification of therapeutic compositions and strategies for the treatment of T1D and/or progression of T1D. More particularly, the present developments include the use of any of the biomarkers of the disclosure as targets to identify test compositions that regulate (up or down) the amount, expression or activity of the biomarker or protein or gene or cell represented by any biomarkers described herein.

These methods are accomplished by phenotyping subjects, e.g. measuring for the presence, absence, increase, decrease, or other changes in particular cellular markers and/or characteristics of these specific cells in response to administration of compositions in accordance with the present developments. These biomarkers and/or characteristics can be detected by any method for analysis of amount or expression of these markers, including, without limitation, cytometry, immunoassay, mass spectrometry, and methods for quantifying proteins and nucleic acids.

Methods of the present developments are based on a study that addressed the impact of Th40 cells, a pathogenic effector subset of helper T cells, in T1D. It should be noted that the Th40 cells discussed herein may occur naturally or may develop as a result of autoinflammation associated with diabetes. In some cases, when CD40 negative cells, including T cells, are exposed to inflammatory conditions, including exposure to the cytokine, interferon-gamma, CD40 is induced on those cells. Thus, in T1D, the cells could arise naturally from bone marrow and thymic development, or may be generated in vivo by inflammation from the disease process.

In this study, Pre-T1D and T1D patients were examined for Th40 cell levels in peripheral blood and the levels were significantly (p<0.0001) elevated compared to controls including healthy non-autoimmune subjects. When the subjects are stratified in Th40 cell levels groups it is evident that several relationships that are paralleled by the preT1D groups 1-4 versus preT1D groups 5-6. There was a significantly higher level of total CD4 T cells in the peripheral blood of T1D and preT1D groups 5-6 subjects compared to control and preT1D groups 1-4, respectively. Moreover, the ratio of CD4 to CD8 was significantly different in control versus T1D and in preT1D groups 1-4 versus 5-6. At any rate, it appears that those subjects with the highest peripheral blood Th40 cell levels are more like T1D subjects immunologically. One note about the cohort used in this study is that the subjects were from a TrialNet study and therefore the subjects have met the criteria for enrollment (HLA haplotype and/or FDR (first degree relative)). That means that in this group there is already a genetic risk factor from the start of the analysis. To that end, the study did find that HLA DR4/DR4 as well as DQ8/DQ8 haplotypes, known to be associated with increased T1D risk, are more represented in the preT1D groups 4-6 than in the preT1D groups 1-3.

In subjects with the highest Th40 cell levels (preT1D groups 5-6), the CD4^{hi} cells produced higher baseline levels of intracellular IL-2 and TNFα. It is possible that this is due to the underlying, ongoing inflammation that may drive the progression of disease development. Interestingly, when T cells from preT1D groups 5-6 were cultured in antigen recall assays the Th40 cells were more likely to produce high levels of IL-4 and IL-17 simultaneously. This profile was present regardless of the presence of APC and antigen, suggesting that those Th40 cells are poised to produce cytokines without any stimulus, other than being removed from the subject and being cultured, which in itself could constitute a stimulus. It may be difficult to interpret what such a cytokine expression profile would mean immunologically since this was done by intracellular staining and essentially is a snapshot in time of what the cells are producing/containing. A number of possible scenarios exist where the cells produce and secrete many cytokines prior to Brefeldin A addition, which would not be detected intracellularly at the time of assaying, or where the cells produce cytokines temporally and therefore, cytokines that are not produced in the 4-hour of Brefeldin A treatment window will not be detected.

Nonetheless, the difference is present and may merit future further analysis to determine what cytokines are secreted and, possibly more important, why the cells from those subjects are so poised to produce cytokines. Certainly, the role of the cytokine environment in T1D remains poorly understood but has moved beyond the Th1/Th2 paradigm. The high IL-17 content found in Th40 cells in the present study may point toward a potential role for IL-17 in preT1D. Several other studies have also found elevated IL-17 in T1D patients. It is highly likely that the total composition and level of all cytokines in the microenvironment will matter.

Peripheral, CD4⁺CD8⁺ double positive (DP) cells were observed in this study. Interestingly, DP cells are elevated in rheumatoid arthritis patients, another autoimmune disease, and are present in the rheumatoid synovium as well (Quandt, D., et al., PLoS One. 2014; 9(3):e93293.) DP cells in rheumatoid arthritis express IL-21, IL-4 and IFNγ and are thought to contribute to the pathogenesis of that disease (Quandt, D., et al., 2014). In this study, the presence of CD4 CD8 DP cells was examined. Typically, DP cells are considered immature, such as those that arise during thymic T cell development. In NOD mice, it was postulated that DP cells escape thymic negative selection and migrate to the periphery (Kishimoto, H., et al., Nature Immunology. 2001; 2(11):1025-1031). These DP cells in preT1D may represent such a population. Additional data may be necessary to determine if DP cells are capable of responding to antigen presented on either MHCI or MHCII, or given the correct microenvironment, both MHCI and MHCII simultaneously.

This study also showed that subjects experiencing impaired glucose tolerance (IGT) have a significantly higher peripheral blood Th40 cell level than controls. This is a direct indication that disease progression in the NOD mouse model, where Th40 cells expand in the prediabetic stages, parallels that of humans.

Additionally, this study found that new onset T1D subjects may have a lower Th40 level than long standing T1D subjects do and there was a trend in subjects with higher number of β-cell associated antibodies towards a lower level of Th40. This may in some instances appear counter intuitive; however, it may align with data because it is known that many T1D subjects experience a honeymoon period recently after the onset of overt hyperglycemia, during which they require less insulin and experience better metabolic control of their blood glucose. Therefore is postulated that the following scenario might occur: 1. A subject at high risk of T1D presents with a high peripheral blood Th40 cell level and one or two autoantibodies. 2. As the subject progresses toward onset, Th40 cells migrate into the affected tissue(s) (pancreatic β-islets) in larger numbers, essentially lowering the Th40 cell level in peripheral blood. During this time, the subject presents with a higher number of autoantibodies. 3. The disease fulminates due to the heavy autoimmune attack and hyperglycemia onsets. This is when the current availability of antigen may have dwindled and therefore the immune attack subsides leading to the honeymoon. 4. Th40 cells, which are long-lived and poised for survival (11,12,27), return to the circulation from the pancreas. Those returning cells, as well as Th40 cells that remained in the circulation, are better poised to be activated than the same cells from a non-autoimmune prone subject. Therefore, those cells begin epitope spreading and eventually rebound to high levels in peripheral blood due to the recognition of new antigens. Th40 cells are especially interesting in such epitope spreading because they are capable of reactivating RAG1 and RAG2 with subsequent TCR alterations.

Considering these data, it may be possible to follow and/or predict disease progression in subjects that demonstrate several risk factors, such as autoantibodies, IGT, and HLA haplotype, together with a high level of Th40 cells. Further, when a dip in the Th40 cell level is observed, overt hyperglycemia may be imminent. Additionally, dips in Th40 cell levels of peripheral blood during overt T1D may occur throughout the disease and such dips may be associated with the onset of complications followed by a “honeymoon” of those symptoms.

Interestingly, the data from this study found that DR4/DR4 haplotypes are more likely to be found in preT1D groups 4-6 (with elevated Th40 cell levels) and that their average age was significantly older than that of all other subjects with any other HLA-DR haplotype. Therefore, additional risk factors of age may be important to predicting the likelihood of developing type 1 diabetes and or the progression thereof. Additionally, an elevated Th40 cell level in association with DR4/DR4 or DQ8/DQ8 as well as two or more autoantibodies may be more predictive of T1D than any of those criteria alone.

Statistical comparisons were made in the study with appropriate linear mixed effect models and variables showing differences ranked by univariate p-value levels in consideration of multiple comparisons with tested variables. The markers indicate drug-target interactions and are useful for, among others, diagnosing, evaluating dosing, evaluating test compounds, evaluating test compositions, and monitoring treatment. Comparisons between cohorts reflect differences that relate to, among others, disease pathophysiology, disease progression, and efficacy of treatment.

One aspect of the present developments includes a method to diagnose a subject as having type 1 diabetes. The method includes the steps of obtaining a sample from a subject, analyzing a subject sample for the percentage of Th40 cells in a sample, comparing the subject sample to a control sample or standard value, and diagnosing type 1 diabetes, an increase in the percentage of Th40 cells in the sample from the subject compared to the control sample or standard value indicating type 1 diabetes in the subject.

In an alternative implementation, the method includes comparison of at least one of the biomarkers in a subject sample with the presence of or amount of one or more of these biomarkers that are present in a sample of a subject known not to have type 1 diabetes. In alternative implementations, additional indicators and/or biomarkers of type 1 diabetes such as, for example those indicators and/or biomarkers of CD4, CD8, CD40, CD25, CD45, TCRV8.3+, CXCR3, CCR5, CD11a, CD11b, CD11d, CD18, CD29, CD41, CD49c, CD49e, CD61, and CD104.

In an alternative implementation, the method may include the detection and measurement of indicators and/or biomarkers of IL-2, IL-4, IL-6, IL-10, IL-17α, IFNγ, and/or TNFα.

In yet another implementation, additional indicators and/or biomarkers of type 1 diabetes, such as, for example, those indicators and or biomarkers known in the art to be associated with T1D, are used in conjunction with the biomarkers of the present developments to diagnose T1D. A preferred sample to test is a serum sample or a whole blood sample.

A preferred change, which may indicate that the subject has T1D, includes an increase in the percentage of Th40 cells in the sample from the subject, relative to a control sample or standard value of about 35%, about 40%, about 45%, about 50%, about 55% about 60%, about 65%, about 70%, about 75%, about 80%, about 85%, about 90%, and about 95%.

The present developments also include a method to diagnose relapse or disease progression in a subject having type 1 diabetes that is being treated for type 1 diabetes. This method includes determining the percentage of Th40 cells in a sample isolated from the subject, comparing the percentage of Th40 cells to a sample or a standard value, and diagnosing relapse or disease progression, the percentage of Th40 cells in the sample from the subject indicating the subject is in relapse or has disease progression.

A change which may indicate that the subject has relapse or disease progression of type 1 diabetes, includes an increase in the percentage of Th40 cells in the sample from the subject, relative to a control sample or standard value of about 35%, about 40%, about 45%, about 50%, about 55% about 60%, about 65%, about 70%, about 75%, about 80%, about 85%, about 90%, and about 95%.

Another aspect of the developments, hereof may also include a dip in the Th40 level may indicate a honeymoon phase or a period of remission of sorts related to the progression of the type 1 diabetes disease.

In yet another implementation, the present developments includes a method to identify a composition useful for the treatment of type 1 diabetes in a subject. The method includes the steps of obtaining an initial sample from a subject, administering a test composition to the subject, obtaining a second sample from the subject after that passage of time, between 1 hrs. and 6 days, and evaluating whether the administration of the test composition causes a transient change in a biomarker. A biomarker can include, CD4, CD8, CD40, CD25, CD45, TCRV8.3+, CXCR3, CCR5, CD11a, CD11b, CD11d, CD18, CD29, CD41, CD49c, CD49e, CD61, and CD104. In this method, a transient change in the biomarker indicates the test composition is useful for treatment of type 1 diabetes. Preferably, such test compositions can be used to further study mechanism associated with type 1 diabetes, serve as a therapeutic agent for use in the treatment or prevention of at least one symptom or aspect of type 1 diabetes, or as a lead composition of such a therapeutic agent.

In yet another implementation, the present developments include a method to identify a dose of a composition, useful for the treatment of type 1 diabetes in a subject, to elicit a desired magnitude of response, including: administering the composition to the T1D subjects at different doses, evaluating the change in the percentage of Th40 cells in a sample isolated from the T1D subjects at the different doses of the composition; and identifying a dose of the composition that elicits the desired magnitude of decrease in the percentage of Th40 cells in samples from the subject over about 1-6 days indicating the test composition is useful for the treatment of T1D.

In yet another implementation, the present developments include a method to identify a dose of a composition, useful for the treatment of type 1 diabetes in a subject, to elicit a desired magnitude of response, including: obtaining a sample isolated from a T1D subject, administering the composition to the T1D subject, evaluating the change in the percentage of Th40 cells in a sample isolated from the T1D subject; and increasing or decreasing the dose of the composition that elicits the desired magnitude of decrease in the percentage of Th40 cells in samples from the subject over about 1-6 days.

One implementation may include determining the levels of autoantibodies to β-cell associated antigens and HLA haplotypes. In some implementations, the reagents needed to determine the levels of β-cell associated antigens and HLA haplotypes may be included in a kit that may further include reagents, vials, syringes, strips, and other materials to determine the level(s) of autoantibodies to β-cell associated antigens. Alternatively, in at least one implementation the levels of autoantibodies to β-cell associated antigens and HLA haplotypes is performed by a laboratory that specializes in determining autoantibodies and HLA haplotypes.

A preferred peptide of the present developments may be one that selectively interacts with a CD40 complex in solution, as determined using an assay such as an immunosorbent assay, or on the surface of a T-cell or other CD40 expressing cells. As used herein, the terms selectively, selective, specific, and the like, indicate the peptide has a greater affinity for a CD40 complex than it does for proteins unrelated to the CD40 complex. More specifically, the terms selectively, selective, specific, and the like indicate that the affinity of the peptide for CD40 is statistically significantly higher than its affinity for a negative control (e.g., an unrelated protein such as albumin) as measured using a standard assay (e.g., ELISA). Suitable techniques for assaying the ability of a peptide to selectively interact with a CD40 complex are known to those skilled in the art. Such assays can be in vitro or in vivo assays. Examples of useful assays include, but are not limited to, an enzyme-linked immunoassay, a competitive enzyme-linked immunoassay, a radioimmunoassay, a fluorescence immunoassay, a chemiluminescent assay, a lateral flow assay, a flow-through assay, an agglutination assay, a particulate-based assay (e.g., using particulates such as, but not limited to, magnetic particles or plastic polymers, such as latex or polystyrene beads), an immunoprecipitation assay, an immunoblot assay (e.g., a western blot), a phosphorescence assay, a flow-through assay, a chromatography assay, a polyacrylamide gel electrophoresis (PAGE)-based assay, a surface plasmon resonance assay, a spectrophotometric assay, a particulate-based assay, an electronic sensory assay and a flow cytometric assay. Methods of performing such assays are well known to those skilled in the art. In one embodiment, an assay can be performed using cells in culture, or it can be performed in a whole animal. Assays can be designed to give qualitative, quantitative or semi-quantitative results, depending on how they are used and the type of result that is desired.

One embodiment of the present developments is a peptide that interacts with a CD40 complex in such a manner as to affect the interaction of the CD40 protein with a CD154 protein, thereby modulating inflammation. The effect of the peptide on the CD40/CD154 interaction can be positive or it can be negative. For example, the peptide can interact with the CD40 complex in such a manner that the strength of the interaction between the CD40 protein and a CD154 protein is increased. Alternatively, the peptide can interact with the CD40 complex such that the strength of the interaction between the CD40 protein and a CD154 protein is decreased. Methods of measuring the strength of binding between the peptide and a CD40 complex are known to those skilled in the art. A preferred peptide of the present developments is one that reduces the strength of the interaction between a CD40 protein and a CD154 protein. Preferred peptides of the present developments reduce the strength of binding between a CD40 protein and a CD154 protein by at least 10%, at least 20%, at least 30%, at least 40%, at least 50%, at least 60%, at least 70%, at least 80%, at least 90%, or at least 95%. A particularly preferred peptide is one that completely inhibits binding of CD40 to CD154. Complete inhibition of binding between CD40 and CD154 means that when a peptide of the present developments is brought into proximity with a CD40 protein and a CD154 protein under conditions that would normally allow the interaction of CD40 and CD154, no such interaction occurs and activation signals are not stimulated in the CD40-expressing cell. Consequently CD40/CD154 mediated modulation of inflammation does not occur. In one embodiment, the peptide interacts with the CD40 protein in such a manner as to reduce the level of inflammation in the system. In one embodiment, the peptide interacts with the CD40 protein in such a manner as to inhibit the development of inflammation in the system.

While peptides of the present developments can interact with any site on the CD40 protein, preferred peptides of the present developments interact with the CD40 complex at a location that overlaps with the CD154 binding site. In one embodiment, a peptide of the present development interacts with the CD40 protein at the CD154 binding site. An example of such a peptide is a CD40 ligand competitive antagonist. As used herein, peptides that interfere with, or inhibit, the binding of a CD154 protein to a CD40 protein are referred to as small interfering peptides (SIPs). As used herein a small interfering peptide is a peptide that, through physio-chemical properties, interferes with the interaction of a CD40 protein with a CD154 protein, thereby preventing activation signals from being delivered to the CD40-bearing cell, thus limiting the activation of the CD40-bearing cell, and consequently, inflammation. As demonstrated herein, the consequences of such interference are prevention of T-cell activation and propagation, and a prevention or reduction of inflammation. Furthermore, one embodiment of the present developments may be a peptide that modulates the CD40-CD154 signaling. In this aspect, the peptide may alter or modulate the CD40-CD154 signaling towards a more anti-inflammatory signal.

A peptide useful for practicing methods of the present developments should be of a size sufficient to interact with CD40 complex in such a manner as to modulate inflammation. It is understood by those skilled in the art that preferred peptides are relatively short since they are easier and less expensive to produce. Preferred peptides are those that are less than 20 amino acids in length. In yet another implementation the developments hereof may include treating a patient that has been diagnosed with type 1 diabetes with a peptide selected from SEQ ID NO: 3, 4, 7, 8, 27, 28, 29, 30, and 32. The sequences of such peptides are shown below in Table 3.

TABLE 3

| SEQ | | |
|---|---|---|
| ID NO | SEQUENCE | Description |
| 1 | MIETYSQPSP RSVATGLPAS MKIFMYLLTV | SwissPro 27548.2 |
| | FLITQMIGSV LFAVYLHRRL DKVEEEVNLH | Mouse CD40 |
| | EDFVFIKKLK RCNKGEGSLS LLNCEEMRRQ | Ligand (CD154 Protein) |
| | FEDLVKDITL NKEEKKENSF EMQRGDEDPQ | |
| | IAAHVVSEAN SNAASVLQWA KKGYYTMKSN | |
| | LVMLENGKQL TVKREGLYYV YTQVTFCSNR | |
| | EPSSQRPFIV GLWLKPSSGS ERILLKAANT | |
| | HSSSQLCEQQ SVHLGGVFEL QAGASVFVNV | |
| | TEASQVIHRV GFSSFGLLKL | |
| 2 | MIETYNQTSP RSAATGLPIS MKIFMYLLTV | SwissPro 29965 |
| | FLITQMIGSA LFAVYLHRRL DKIEDERNLH | Human CD40 |
| | EDFVFMKTIQ RCNTGERSLS LLNCEEIKSQ | Ligand (CD154 Protein) |
| | FEGFVKDIML NKEETKKENS FEMQKGDQNP | |
| | QIAAHVISEA SSKTTSVLQW AEKGYYTMSN | |
| | NLVTLENGKQ LTVKRQGLYY IYAQVTFCSN | |
| | REASSQAPFI ASLCLKSPGR FERILLRAAN | |
| | THSSAKPCGQ QSIHLGGVFE LQPGASVFVN | |
| | VTDPSQVSHG TGFTSFGLLK L | |
| 3 | KGYY | Core-sequence |
| 4 | KKGYYT | 6-mer |
| 5 | AKKGYYTM | 8-mer-mouse |
| 6 | AEKGYYTM | 8-mer human |
| 7 | VLQWAKKGYYTMKSN | 15-mer-mouse |
| 8 | VLQWAEKGYYTMSNN | 15-mer human |
| 9 | NAASVLQWAKKGYYTMKSNLVMLE | 24-mer mouse |
| 10 | ISQAVHAAHAEINEAGR | 15-mer from ovalbumin; |
| | | control peptide |
| 11 | G-L-Q-W-A-K-K-G-Y-Y-T-M-K-S-N | Gly-1 |
| 12 | V-G-Q-W-A-K-K-G-Y-Y-T-M-K-S-N | Gly-2 |
| 13 | V-L-G-W-A-K-K-G-Y-Y-T-M-K-S-N | Gly-3 |
| 14 | V-L-Q-G-A-K-K-G-Y-Y-T-M-K-S-N | Gly-4 |
| 15 | V-L-Q-W-G-K-K-G-Y-Y-T-M-K-S-N | Gly-5 |
| 16 | V-L-Q-W-A-G-K-G-Y-Y-T-M-K-S-N | Gly-6 |
| 17 | V-L-Q-W-A-K-G-G-Y-Y-T-M-K-S-N | Gly-7 |
| 18 | V-L-Q-W-A-K-K-G-G-Y-T-M-K-S-N | Gly-9 |
| 19 | V-L-Q-W-A-K-K-G-Y-G-T-M-K-S-N | Gly-10 |
| 20 | V-L-Q-W-A-K-K-G-Y-Y-G-M-K-S-N | Gly-11 |
| 21 | V-L-Q-W-A-K-K-G-Y-Y-T-G-K-S-N | Gly-12 |
| 22 | ISQAVHAAHAEINEAGR | 15-mer from ovalbumin; |
| | | control peptide |
| 23 | YVQGKANLKSKLMYT | Scrambled peptide |
| 24 | WAKKGYYTMK | 10-mer mouse |
| 25 | VLQWAKKGYYTMK | 13-mer mouse |
| 26 | AASVLQW AKKGYYTMKSNLVMLEN | 24-mer mouse |
| 27 | KGYYTM | 6-mer (Form 2) human |
| 28 | AEKGYY | 6-mer (Form 3) human |
| 29 | AKKGYY | 6-mer (Form 4) mouse |
| 30 | AKGYYT | 6-mer (Form 5) synthetic |
| 31 | YKNVKQMAYWLTGKS | Scrambled peptide |
| 32 | AKKGYY + Val | 6-mer (Form 6) |
| | | mouse +Val |

In some instances, the implementations of the developments herein may reference peptides developed for the treatment of disease which are further described in U.S. Pat. No. 9,562,088 the entire disclosure of which is hereby incorporated by reference herein in its entirety by this reference thereto for all that this prior document discloses as if fully set forth here. In other implementations, the developments herein may reference peptides for the treatment of disease which are further described in U.S. application Ser. No. 16/184,129, U.S. application Ser. No. 16/240,630, and U.S. Prov. App. No. 62/821,941 the entire disclosures of which are hereby incorporated by reference herein in their entireties by this reference thereto for all that the these prior documents disclose as if fully set forth here.

### EXAMPLE S

This study addresses the impact of Th40 cells, a pathogenic effector subset of helper T cells, in pre-T1D and T1D subjects.

An identified CD4 T cell subset, characterized by the expression of CD40 (Th40 cells), which is significantly (p<0.0001) expanded in number in peripheral blood from T1D patients but not from non-autoimmune control or non-autoimmune T2D patients (9). That expansion was present in T1D patients regardless of HLA haplotype and, in a blinded study, we successfully identified T1D patients versus control subjects with 95% accuracy. Similar to our findings in human T1D patients, we found an expansion of Th40 cells in the non-obese diabetic (NOD) mouse model of T1D (10-14). Unlike humans, T1D in NOD mice runs a consistent, predictable course. In those mice, Th40 cells progressively expand in the prediabetic stages (3-12 weeks of age), concurrently with establishment of insulitis (10-14). In very young NOD mice, in the pre-insulitis stage, peripheral Th40 cells are at numbers equivalent to controls, but expansions already occur in pancreatic lymph nodes (15). Eventually overt hyperglycemia ensues, at which stage peripheral Th40 cell numbers reach up to 60% compared to about 10-15% in control mice (10,12). Th40 cells are also necessary and sufficient to transfer T1D from both prediabetic (with present insulitis) and diabetic NOD mice to NOD.scid mice (10,12,14). Interestingly, Th40 cells are capable of reactivating recombination activating gene (RAG) 1 and 2 which leads to alteration of the TCRs expressed by these mature, peripheral T cells (16,17). Such alterations could potentially lead to epitope spreading, thus expanding the TCR repertoire. In recent work, we demonstrated that a peptide that targets the CD40 molecule could prevent development of diabetes and, more importantly, reverse overt hyperglycemia in almost 60% of diabetic mice if treated immediately after reaching hyperglycemic levels (18). Translationally, this would correspond to new onset in human T1D.

Because of these findings, this study and these examples set out to assess Th40 cell levels in prediabetic subjects (preT1D) enrolled in the TrialNet study. TrialNet recruits subjects based on genetic risk factors (HLA haplotype) as well as being a first-degree relative (FDR) of patients with T1D. Here we show that when preT1D subjects are stratified according to peripheral blood Th40 cell percentages, several relationships between preT1D subjects with low and high Th40 cell levels parallel the relationships that are seen between control and T1D subjects. This type of stratification also reveals cytokine production, CD4/CD8 double-positive population, and HLA-DR differences between the preT1D groups where significant patterns emerge. Because Th40 cells are readily detected in peripheral blood, these observations suggest a useful biomarker that, in association with other risk factors, may predict diabetes risk.

Materials and Methods

Subjects were recruited at the Barbara Davis Center for Diabetes (BDC) and were consented under COMIRB protocol # 01-384. TrialNet criteria for inclusion were high risk HLA and/or first degree relative of a subject with T1D.

Antibody Titers and HLA Determination.

Levels of autoantibodies to β-cell associated antigens and HLA haplotypes were determined at the Barbara Davis Center Autoantibody/HLA Service Center, which is a CLIA certified laboratory and has been designated as a NIH/NIDDK North America Autoantibody/HLA Core Laboratory.

Oral Glucose Tolerance Test (OGTT).

OGTT was performed at the Barbara Davis Center clinic. A two-hour 75-gram oral glucose tolerance test (OGTT) was performed. The subjects were asked to arrive at the clinic fasting for at least eight hours. A healthcare provider performed a blood glucose test to determine the fasting glucose level then administered 8 ounces of a solution containing a total of 75 grams of glucose, orally, over a five-minute period. Two hours after ingestion of the glucose solution, another blood glucose test was performed. A blood glucose level at 2 hours of less than 140 mg/dl was considered normal, a level between 140 and 199 mg/dl was considered impaired glucose tolerance, and a level of 200 mg/dl or greater was considered diabetic. Isolation of peripheral blood mononuclear cells (PBMC).

Blood samples were collected into sodium-heparin vials by venipuncture. The blood was processed within 2-6 hours of collection using Ficoll-Paque PLUS (GE Healthcare, Pittsburgh, Pa.) density gradient media according to the manufacturer's instructions. This yields 95±5% mononucleocytes, 3±2% granulocytes, 5±2% erythrocytes, and less than 0.5% of total platelets of the original blood sample remain.

Cell Staining and Flow Cytometry.

PBMC, 150,000-500,000 cells, were stained in 100 μl Phosphate Buffered Saline containing 2 mM EDTA and 0.5% bovine serum albumin (running buffer). Antibodies were added at 0.2-0.5 μg each and incubated for 20-30 minutes at room temperature. Cells were washed then fixed in 2% paraformaldehyde. If intracellular staining was performed, cells were resuspended in 100 μl eBioscience™ permeabilization buffer (Thermo Fisher Scientific, Waltham, Mass.) then antibodies were added at the concentration above and incubated for 30-45 minutes at room temperature. Cells were washed and run on a MACSQuant® Analyzer (Miltenyi Biotec Inc., Auburn, Calif.). Staining was done in the presence of Fc-receptor block (Thermo Fisher Scientific, Waltham, Mass.). Antibodies were: Anti-CD40 (G28-5; produced in-house and conjugated to Alexa-Fluor 405). Anti-CD3 (UCHT1; PerCp-Cy5.5), anti-CD4 (RPA-T4; APC-Cy7), and anti-CD8 (OKT8; APC) all from Tonbo Biosciences, San Diego, Calif. Anti-CD45RO (UCHL1; PE), anti-IL-2 (MQ1-18H12; PE), anti-IL-4 (8D4-8; PE-Cy7), anti-IL-6 (MQ2-13A5; PE), anti-IL-10 (JE53-9D7; PE), anti-IL17α (eBio64DEC17; APC), anti-IFNγ (45.B3; FITC), and anti-TNFα (Mab 11; PE) all from Thermo Fisher Scientific, Waltham, Mass. Data was analyzed using FlowJo analysis software. Cells were gated on FSC vs SSC to gate out debris and dead cells, and then gates were set based on isotype controls. Cells were gated for CD4⁺CD40⁺ (Th40 cells), CD4⁺CD40⁻ (CD4hi T cells) or CD8⁺and CD3 expression was confirmed. CD45RO expression was assessed within the gated Th40 and CD4hi T cells. For intracellular cytokines, gates were set based on isotype control as well as the CD4⁻CD40⁻ population (lower left quadrant) from each sample as a negative control. This population does not demonstrate any significant intracellular cytokine expression and serves as a built-in control for the actual antibodies used to stain the sample.

Antigen Recall Assay.

Purified PBMC were sorted on HLA-DR⁺IgD⁺/CD11b⁺ using microbeads (Miltenyi Biotec Inc., Auburn, Calif.) to yield antigen presenting cells (APC). The remaining cells were considered T cells. Cells were resuspended in AIM-V® medium containing 10% fetal calf serum (Thermo Fisher Scientific, Waltham, Mass.). APC, 1×10⁵ cells in 100 μl, were plated in a round bottom 96-well tissue culture plate. A final concentration of 20 μM peptide antigen (PLP₁₀₃₁₂₀, MBP₈₃₋₁₀₂, GAD₅₅₆₋₅₇₀, GAD₂₇₁₋₂₈₅, Pro-insulin peptide, or Insulin B₉₋₂₃) was added. Additionally, 20 islets from a diabetic patient were used as an antigen or, as a control, 2 ul of Pentacel vaccine (reconstituted according to the manufacturer; Sanofi Pasteur, Swiftwater, Pa.) was added. Cells were incubated for 2 hours at 37° C. then 100 μl T cells were added at the same ratio that was found in the to be present in the particular subject during the sort. That is, if the APC:T cell ratio was 1:5, then 5×10⁵ T cells were added; if the ratio was 1:3 then 3×10⁵ T cells were added etc. Incubation was continued overnight then Brefeldin A (Sigma-Aldrich, St. Louis, Mo.) was added to a final concentration of 5 μg/ml. Cells were incubated another 4 hours in the presence of Brefeldin A then the cells were stained for Th40 cells and cytokine content as above.

Western Blot.

Cells were sorted using anti-CD4- or anti-CD8-conjugated microbeads followed by magnetic sort on an AutoMACS (Miltenyi Biotec Inc., Auburn, Calif.). Western blot was performed according to Laemmli, loading 10 ug protein per lane of 10% TGX gels and transferring to PVDF membrane (Bio-Rad Laboratories, Hercules, Calif.). Antibodies used for western blot were anti-CD4 (C-18; sc-1140, Santa Cruz Biotechnology, Dallas, Tex.) and anti-CD8 (PAS-11453, Thermo Fisher Scientific, Waltham, Mass.). As an internal standard, the membranes were stripped and stained with Coomassie blue R-250 to visualize all the protein on the membrane. A representative band was used in the figure.

Results

PreT1D Subjects Can Be Stratified by Th40 Cell Level Standard Deviation Groups.

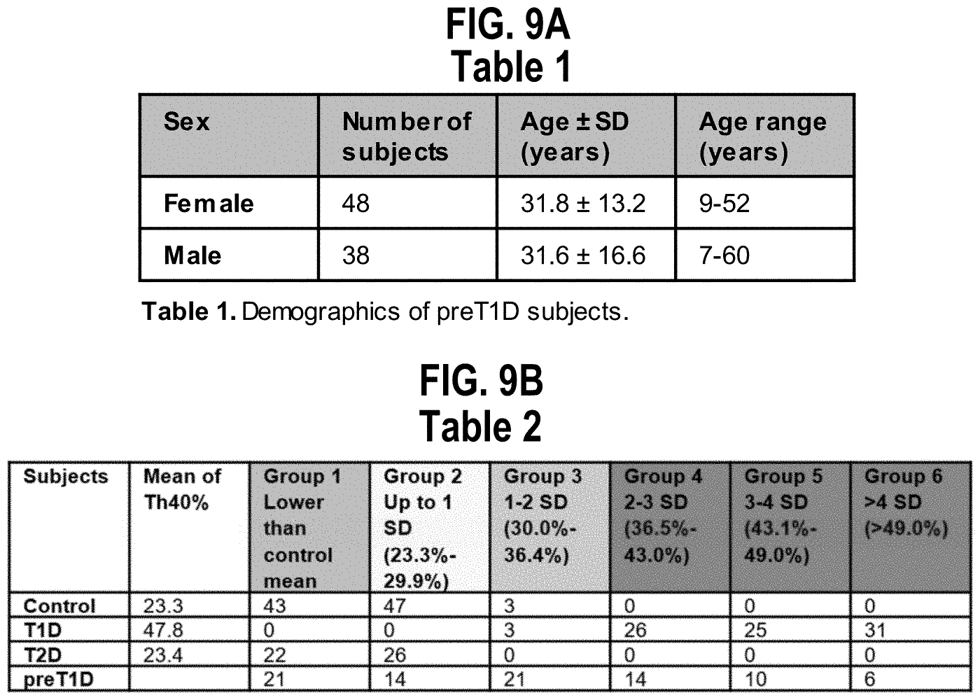

In previous work, we showed that T1D subjects have significantly elevated levels of Th40 cells in peripheral blood compared to control subjects and that elevation is present regardless of HLA-haplotype (9). HLA genes are the most consistent genetic markers for T1D, yet not every subject with an at-risk HLA becomes diabetic. Likewise, not all T1D subjects have the high risk HLAs. Therefore, the Th40 cell level may be a better biomarker for risk of developing T1D. We acquired blood samples from preT1D subjects enrolled in the TrialNet study (Table 1). Those demographics did not differ from the demographics published previously for our T1D, T2D, and control subjects (9). From the original T1D patient data (9), we created a table of relative Th40 cell levels (Table 2—FIG. 9B). Using the determined mean and standard deviation (SD) of the Th40 cell level from non-autoimmune control subjects, we generated Th40 relative groups by cumulatively adding the control SD to the control mean. The groups were: Group 1—any value lower than control mean, Group 2—control mean+up to 1 SD, Group 3—control mean+1-2 SD, Group 4—control mean+2-3 SD, Group 5—control mean+3-4 SD, and Group 6—control mean+>4 SD. Based on these groups we ranked the samples from control, T1D, and T2D subjects (Table 2—FIG. 9B). As expected, T1D subjects clearly, and almost exclusively, fell into groups 4, 5, and 6. Also as expected, non-autoimmune T2D subjects fell into groups 1 and 2, the same as the control subjects (Table 2—FIG. 9B). When we applied these groupings to preT1D subjects, 35% of the subjects fell into groups 4-6, with over half of those in groups 5 and 6. Therefore, assuming that preT1D subjects can be divided into those that will develop T1D and those who will not, and that those groups will be defined by higher versus lower peripheral blood Th40 cell levels, we studied the relationship between the groups and compared it to the relationship between control and T1D subjects.

Stratified PreT1D Subjects Parallel Several Relationships Found in Control Versus T1D.

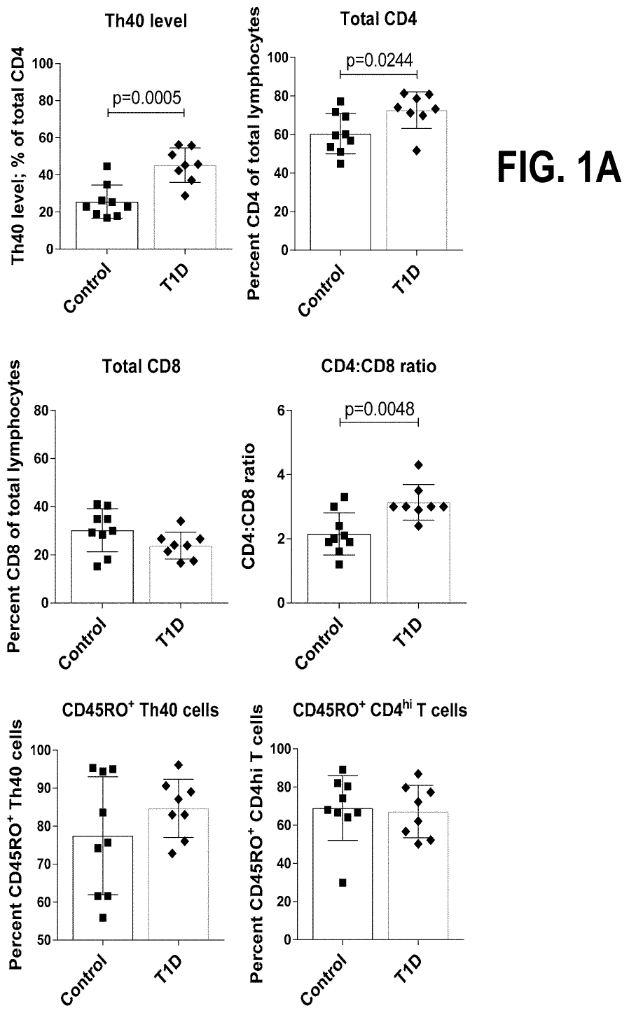

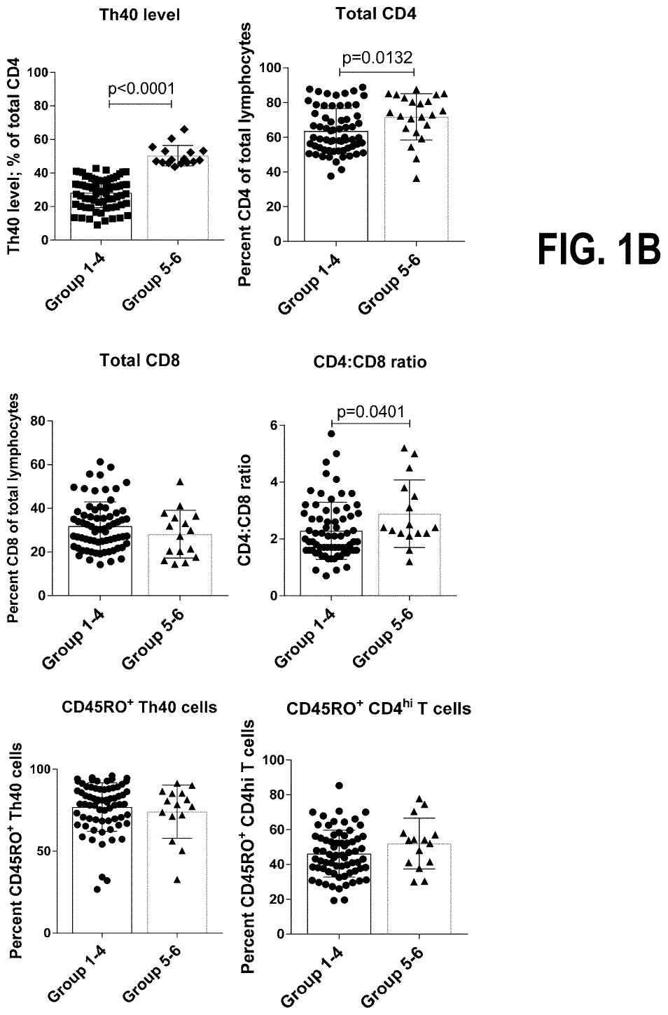

T1D subjects consistently have a significantly higher level of Th40 cells in peripheral blood compared to control subjects (FIG. 1A; p=0.0005; two-tailed t-test) as reported previously (9). Th40 cells often demonstrate a lower surface expression of CD4 (9) while the overall expression, surface and intracellular, is similar to that of conventional CD4-high expressors (11). We analyzed CD4 and CD8 levels, taking care not to gate out the CD4-low expressors, and found significant differences between T1D and control subjects in CD4 percentages and CD4:CD8 ratio (FIG. 1A; p=0.0244 and p=0.0048, respectively; two-tailed t-test). Because almost all control subjects fell into groups 1-3 and almost all T1D subjects fell into groups 4-6 (Table 2—FIG. 9B) we compared preT1D subjects in groups 1-3 with those in groups 4-6. Because we grouped based on Th40 cell level, the Th40 cell level means were necessarily significantly different but when analyzing other parameters we found no other significant differences when grouping this way. Therefore, we applied a more stringent grouping, comparing preT1D subjects in groups 1-4 to those in groups 5-6. Again, the Th40 cell levels were necessarily significantly different (FIG. 1B; p<0.0001; two-tailed t-test). The CD4 T cell content of total lymphocytes, in groups 5-6 was significantly greater than that of groups 1-4 (FIG. 1B; p=0.0132; two-tailed t-test), paralleling the relationship between T1D and control subjects (FIG. 1A). When the same analysis was done for total CD8 T cell content, no significant difference was found between control and T1D or preT1D groups 1-4 and 5-6, although there was a trend towards a lower level in T1D as well as in preT1D groups 5-6 (FIG. 1A and FIG. 1B). We then compared the CD4:CD8 ratio and found that preT1D groups 5-6 had a significantly higher ratio than groups 1-4 (FIG. 1B; p=0.0401; two-tailed t-test), again paralleling the T1D/control relationship. Th40 cells tend to express lower cell surface levels of CD4 (CD4lo) (9), which has long been known to be associated with an activated T cell state (19). T1D is also suggested to be due to the persistence of memory T cells that attack β-islet cells (20). Therefore, we examined the memory marker CD45RO in CD4lo (Th40 cells) and CD4hi (conventional CD4) T cells. We did not discover any significant difference between control and T1D or preT1D groups 1-4 and 5-6 (FIG. 1A and FIG. 1B).

Stratifying PreT1D Subjects According to Th40 Cell Levels Reveals Differences in Cytokine Production Potential.

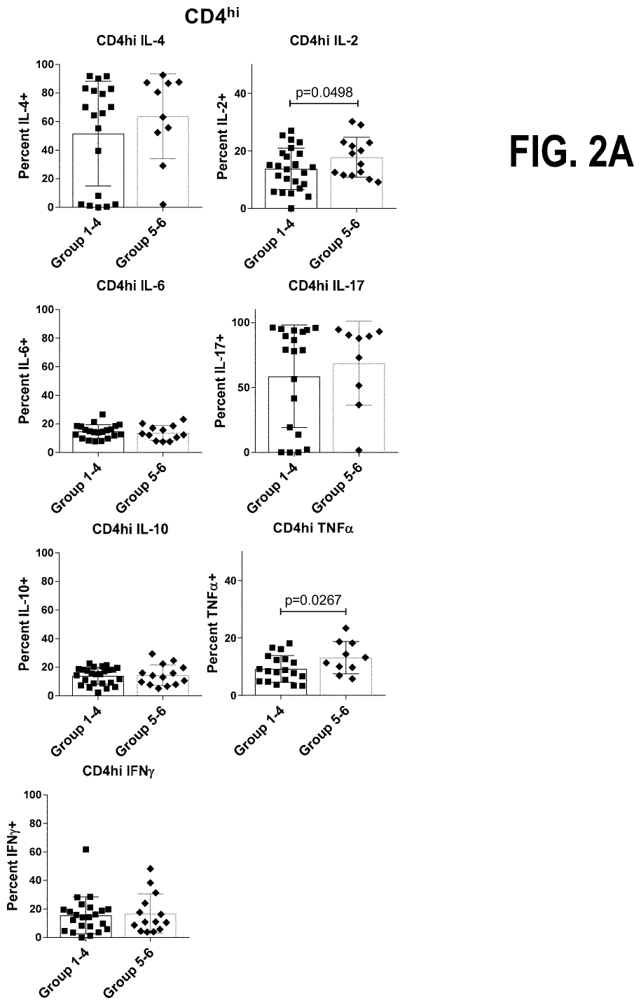

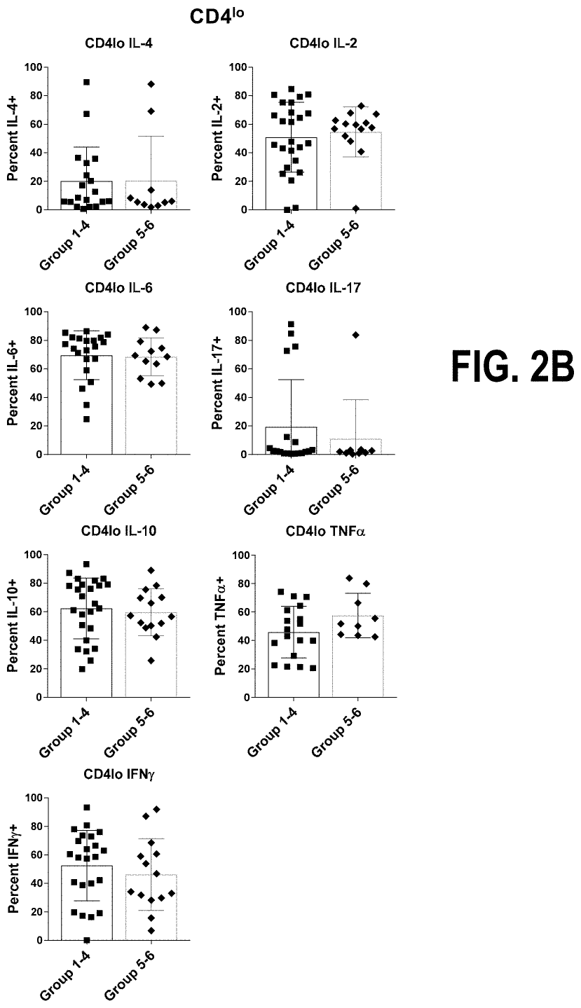

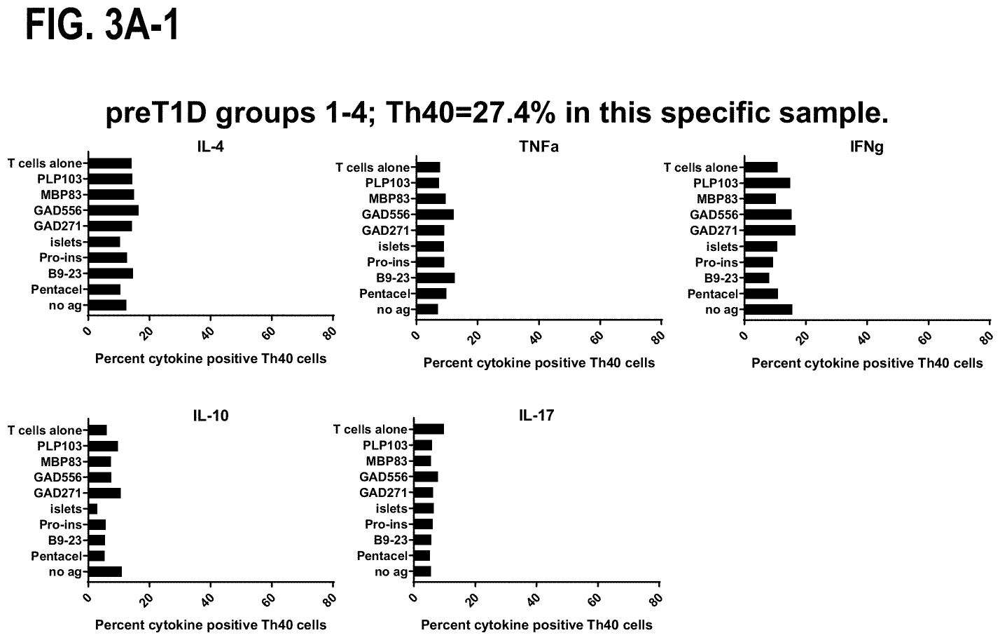

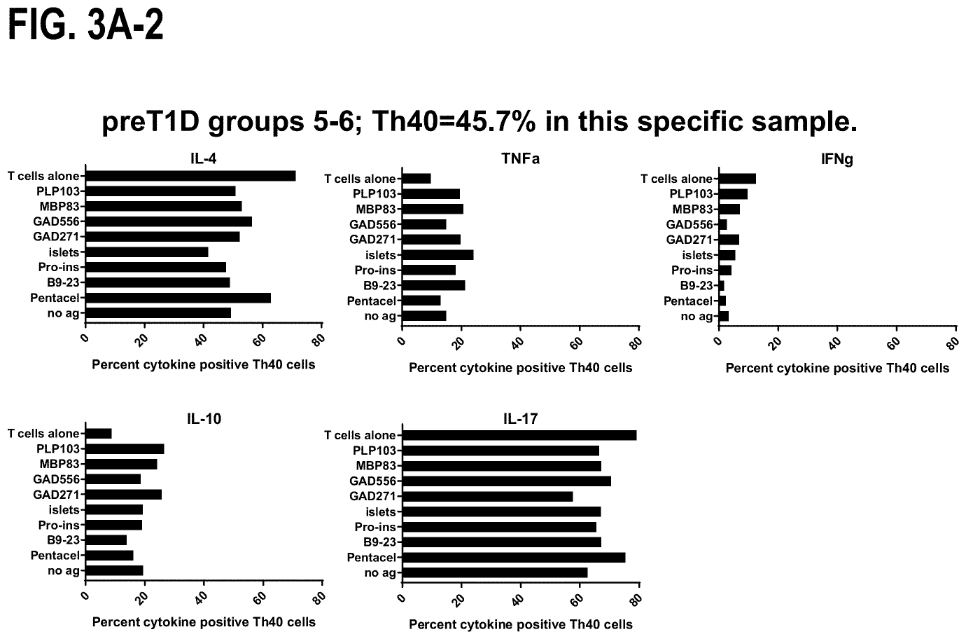

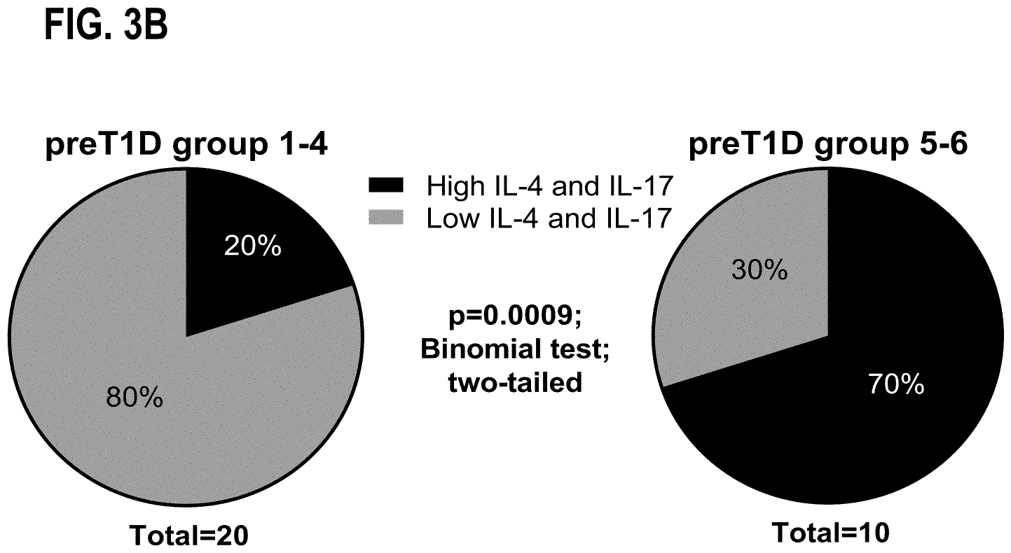

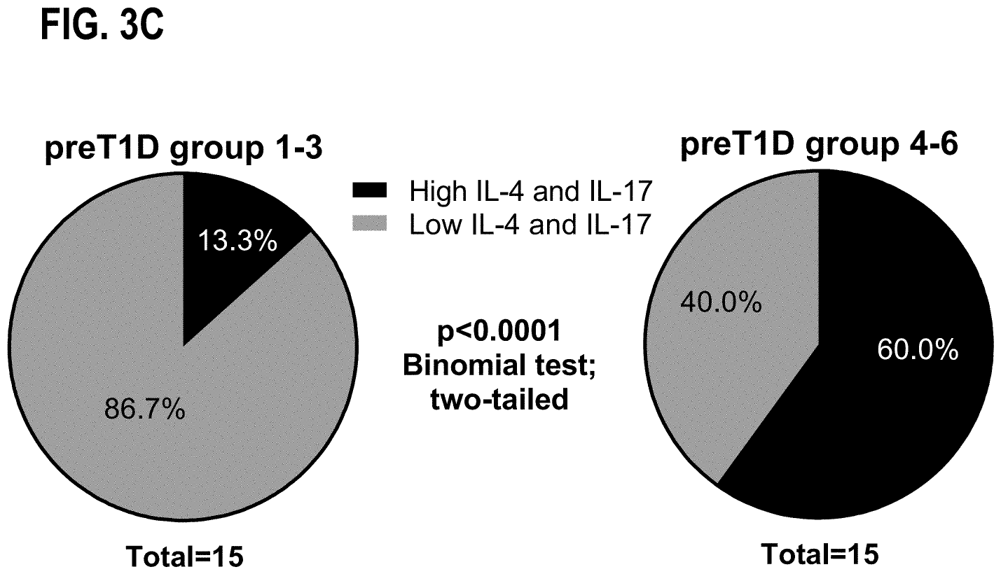

Given the autoimmune nature of T1D, an underlying, ongoing, inflammation may not only drive the disease but also cause its fulmination. Therefore, we examined both baseline cytokine expression and antigen-recall cytokine expression by intracellular staining in a subgroup of the preT1D subjects. For the baseline cytokine, we examined peripheral blood mononuclear cells (PBMCs) immediately after isolation. Since Th40 cells generally express a lower level of CD4 on their surface, we gated on CD4lo and CD4hi T cells (conventional CD4 T cells) in preT1D groups 1-4 and 5-6 and analyzed intracellular cytokine levels (FIG. 2A and FIG. 2B). Few differences were detected, but CD4hi T cells from groups 5-6 expressed more IL-2 and TNFα than CD4hi T cells from groups 1-4 (FIG. 2A and FIG. 2B; p=0.0498 and p=0.0267, respectively; two-tailed t-test). We then performed antigen-recall assays and graphed the intracellular expression levels of IL-4, TNFα, IFNγ, IL-10, and IL-17, in CD4lo (Th40) cells from preT1D groups 1-4 and 5-6. Interestingly, a pattern emerged where 70% of the subjects in preT1D groups 5-6 had high levels of intracellular IL-4 and IL-17 regardless of antigen or the presence of antigen presenting cells (FIG. 3A-1, FIG. 3A-2 and FIG. 3B). This was true for only 20% of the subjects in preT1D groups 1-4 (FIG. 3A-1, FIG. 3A-2 and FIG. 3B). The difference between the two groups was significant (p=0.0009; two-tailed binomial test). If we set the cutoff at 2SD instead (groups 1-3 versus groups 4-6), the significant difference was still present (FIG. 3C; p<0.0001; two-tailed binomial test)

The Presence of a CD4/CD8 Double Positive Population is More Common in PreT1D Groups 5-6.

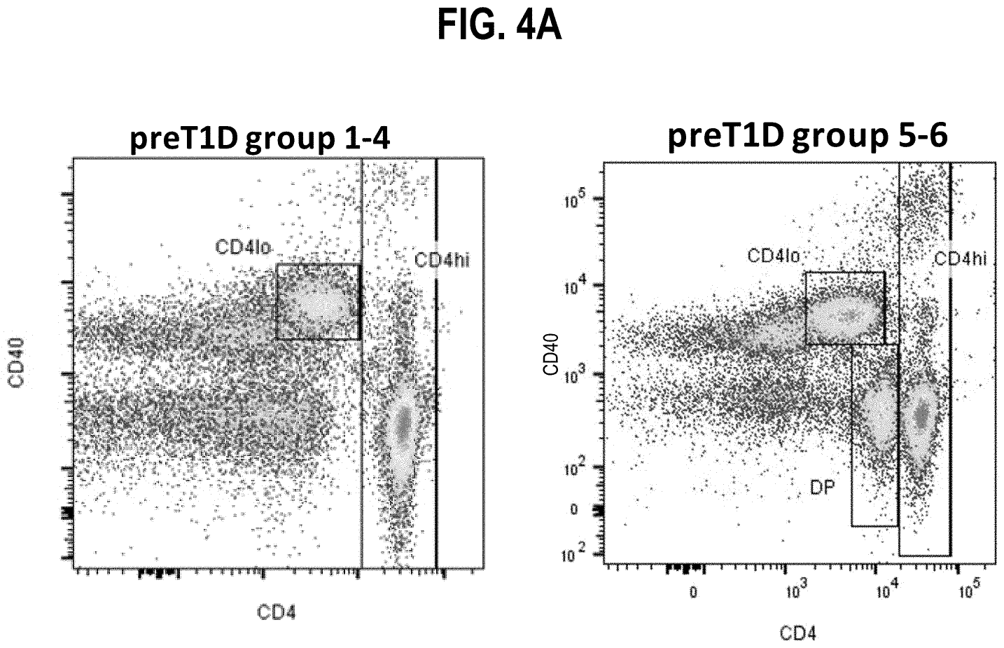

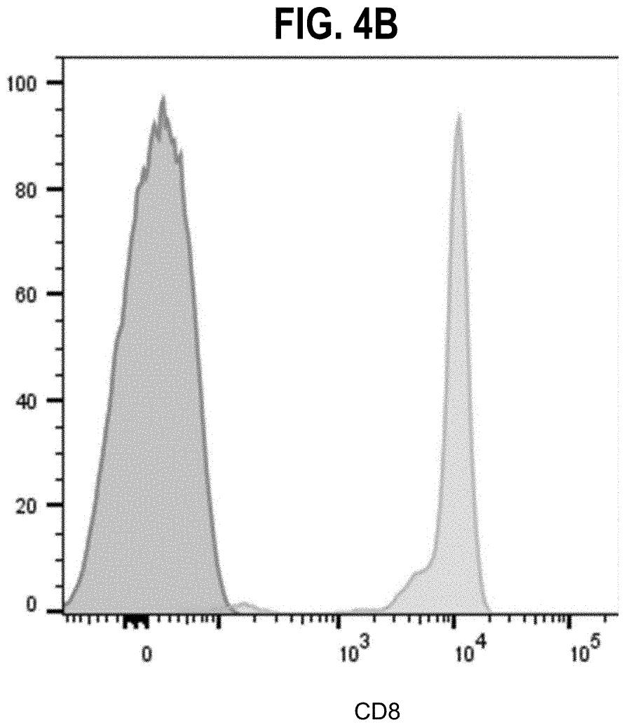

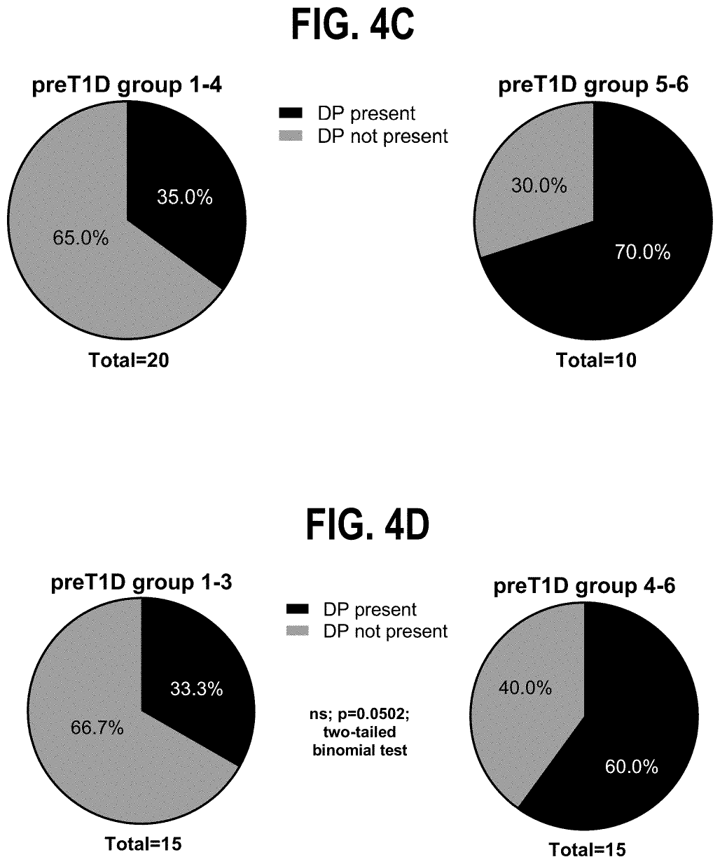

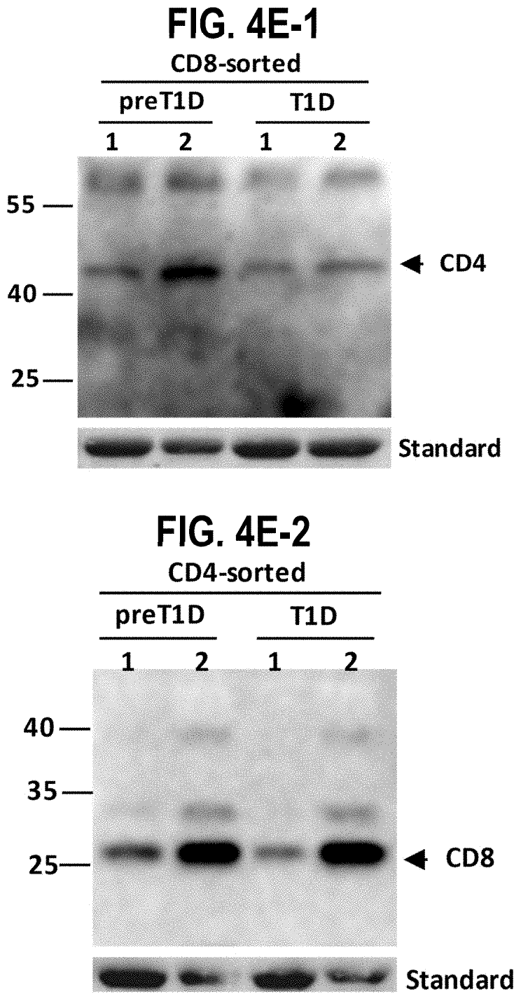

When plotting CD4 versus CD40 in stains of the PBMC from preT1D subjects, we noticed that in some samples a population that was CD4-intermediate was present (see, for example, FIG. 4A, and FIG. 4E-1). When further analyzing that population it became clear that it was not only CD4-positive but was also positive for CD8 (FIG. 4A, right panel, and FIG. 4B). Interestingly, that double-positive population was significantly more prevalent in the preT1D groups 5-6 than in groups 1-4 (FIG. 4C; p=0.0395; two-tailed binomial test). If the cutoff was set to 2 SD (groups 1-3 versus 5-6), the significant difference was no longer apparent (FIG. 4D; p=0.0502; two-tailed binomial test). Further confirming double-positivity, we sorted CD8 or CD4 T cells from PBMCs from preT1D and T1D subjects that stained positive for that population and performed western blots for the molecule not sorted on, i.e. if sorted on CD4, western was done for CD8 and vice versa (FIG. 4E-1 and FIG. 4E-2). Very clearly, cells sorted on CD8 then probed for CD4 demonstrated CD4 protein expression and cells sorted on CD4 demonstrated CD8 expression (FIGS. 4E-1 and 4E-2) demonstrating that the double-positive population was not an artefact of staining in flow cytometry.

Th40 Level Does Not Correlate with Age or Antibody Status.

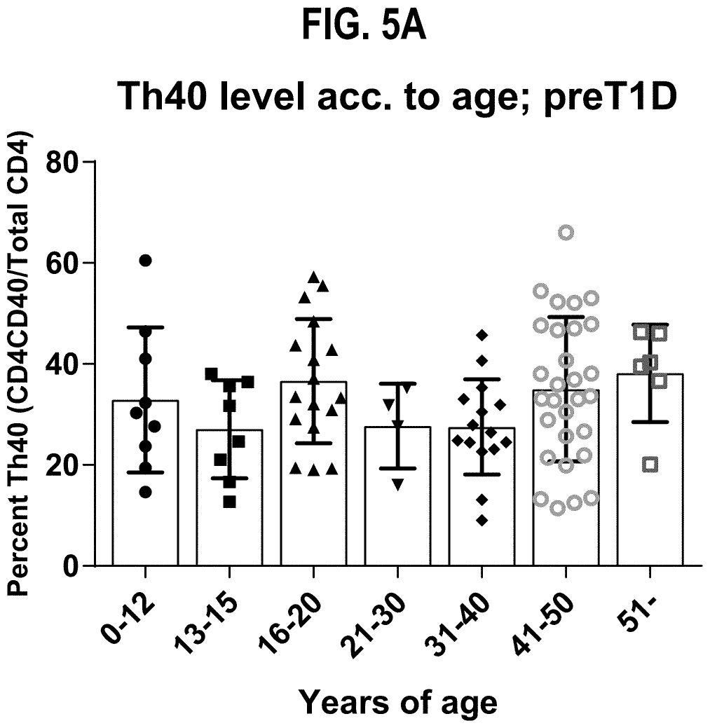

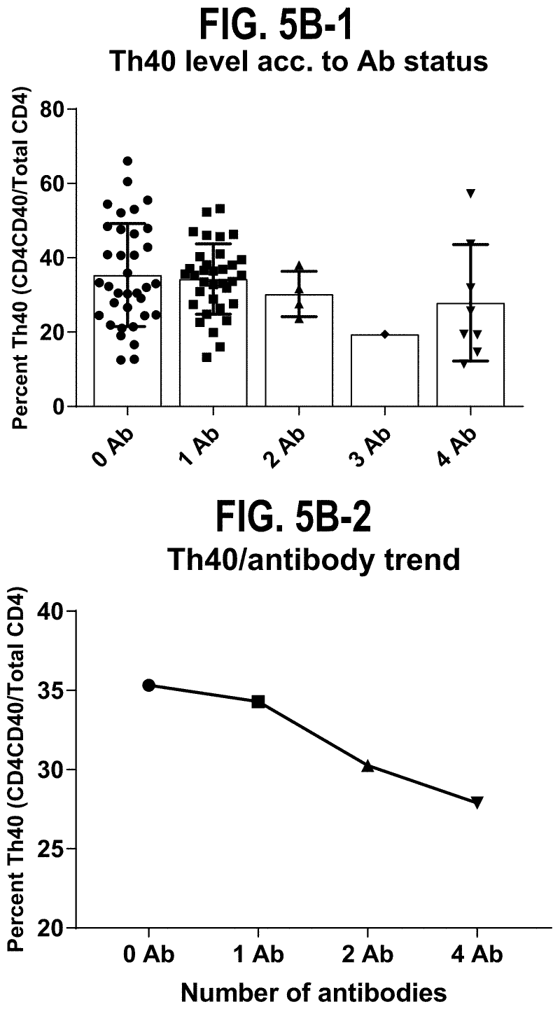

Previously we demonstrated that Th40 cell level does not correlate with age (9). As that data was from the general population, controls and T1D subjects without any further discrimination, and we here have subjects enrolled in TrialNet because of known risk factors, HLA and/or first degree relative of a T1D subject, we performed the same analysis on this more narrowly defined cohort. Similar to what we found before, there was no significant difference in Th40 cell levels between different age groups and a highly variable range was observed in each age group (FIG. 5A).

The presence of islet autoantibodies in the serum of preT1D subjects is considered a risk factor for developing T1D (21). Therefore, we analyzed Th40 cell levels based on autoantibody status among the preT1D subjects. There was no significant difference in Th40 cell level based on antibody (Ab) status (FIG. 5B-1). In fact, the Th40 cell level range was quite broad for the 0, 1, and 4 Ab groups; range 12-66%, 13-53%, and 11-57%, respectively (FIG. 5B-1). In subjects with 2 Ab the range was more narrow, 24-38%, but there were only 4 subjects in this group (FIG. 5B-1). In addition, we only had one subject with 3 Ab. Regardless of the range, there was a trend toward a lower level of Th40 cells as the number of antibodies increased (FIG. 5B-2).

PreT1D Subjects with Impaired Glucose Tolerance Have Higher Levels of Peripheral Blood Th40 Cells Than Control Subjects Do.

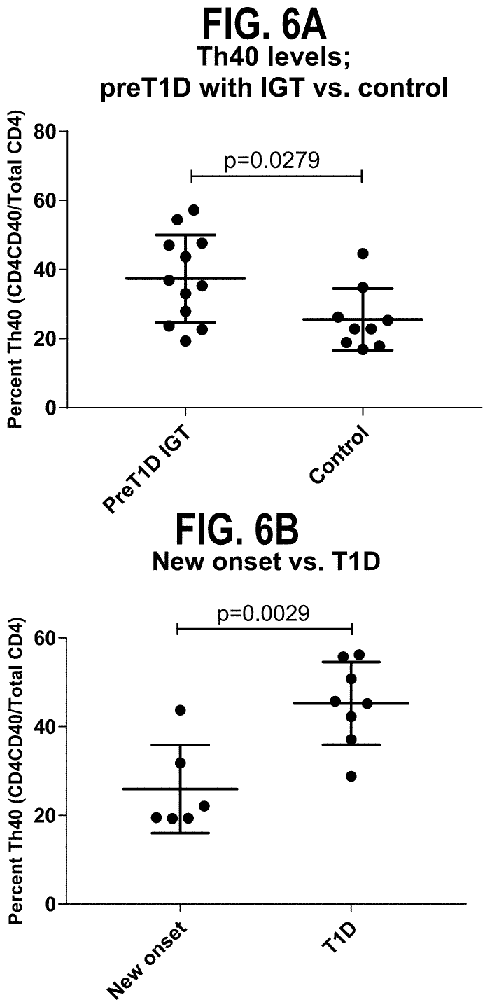

Impaired glucose tolerance (IGT) precedes overt hyperglycemia. At the time when oral glucose tolerance tests were performed, we acquired blood samples from the preT1D subjects for Th40 cell level determination. When peripheral blood Th40 cell levels were analyzed it was revealed that subjects experiencing IGT had significantly higher levels of Th40 cells compared to control subjects (FIG. 6A; p=0.0279; two-tailed t-test).

New Onset T1D Subjects Experience a Dip in the Peripheral Blood Th40 Cell Level.

Many new onset T1D subjects experience a honeymoon period during which they require less insulin and have better glycemic control (4). The honeymoon phase is thought to be influenced by both metabolic and immunological factors and is regarded as one of many phases of remission occurring during the disease (4). We acquired several samples from subjects that had recently, within 6 months, onset with T1D. Analysis of the peripheral blood Th40 cell levels revealed that this group typically had significantly lower levels of Th40 cells than those of long standing T1D subjects (FIG. 6B; p=0.0029; two-tailed t-test).

Can Trends Be Discerned That May Predict when a PreT1D Subject Will Become Overtly Hyperglycemic?

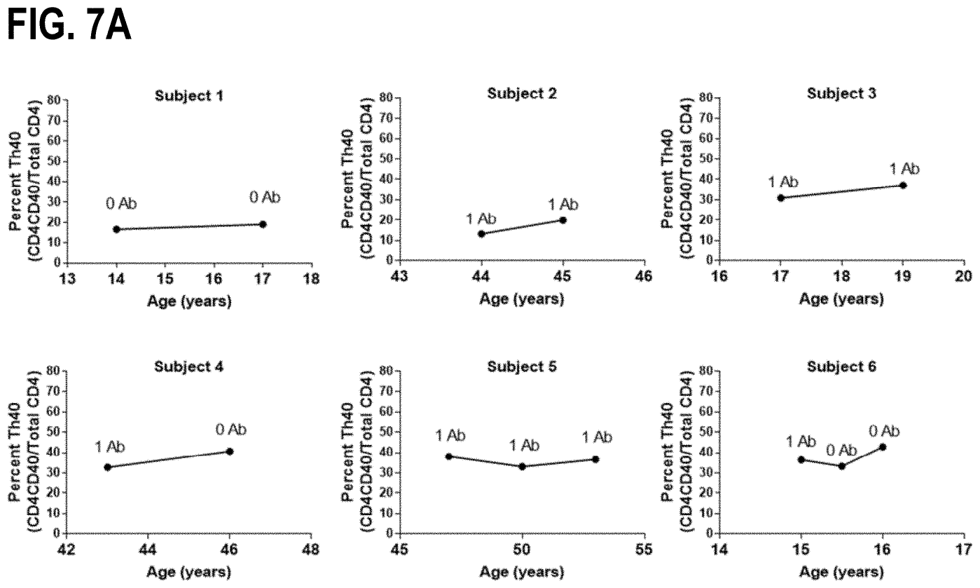

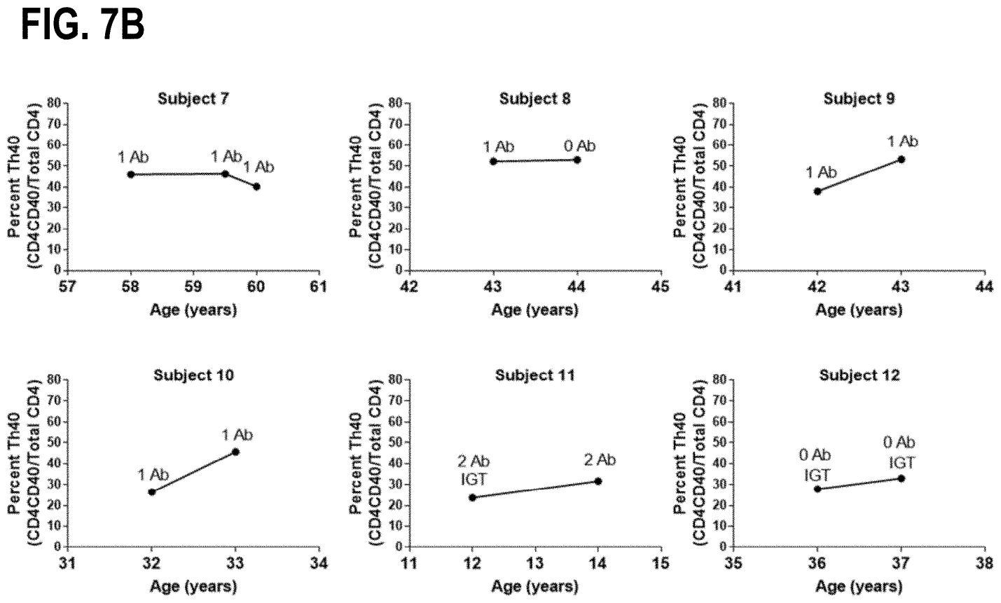

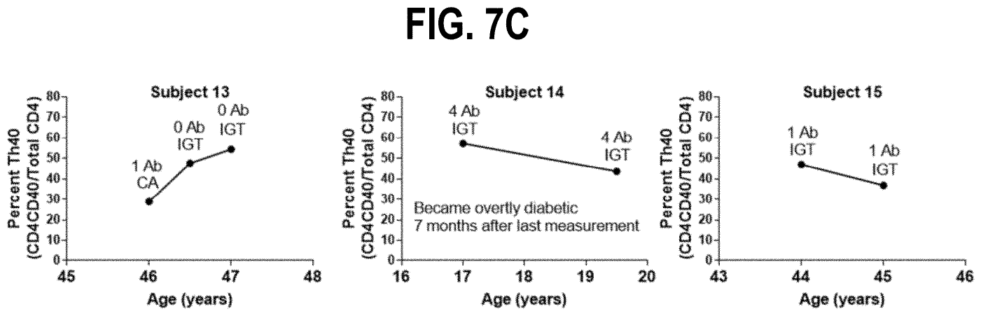

For some preT1D subjects we were able to perform longitudinal analysis on Th40 cell status. When viewing the graphs of these subjects' Th40 cell levels over time, several patterns emerged (FIGS. 7A, 7B, and 7C). There were those that had levels comparable to controls and remained stable at those levels and there were those that were at higher Th40 cell levels but remained stable at those levels. Several subjects started at a low to intermediate Th40 cell level but increased sharply over time while others started at a high level but decreased sharply over time. In the last group, one subject was diagnosed with T1D seven months after the last Th40 cell measurement. That subject also was positive for four (3-cell antigen antibodies. This is interesting in the light of the above data, which demonstrates that 4 Ab positive subjects trend toward a lower level of peripheral blood Th40 cells and that new onset T1D subjects have lower levels of peripheral blood Th40 cells compared to long standing T1D subjects. Several subjects in the latter two groups (sharp increase or decrease) experienced IGT at the time of sample collection. Therefore, it is possible that subjects that sharply expand their Th40 cell levels over time and then experience a sharp decrease are those that should be considered at highest risk of imminent hyperglycemia.

PreT1D Subjects with HLA DR4-DR4 Haplotype Are More Likely to be Found in Th40 Level Groups 4-6.

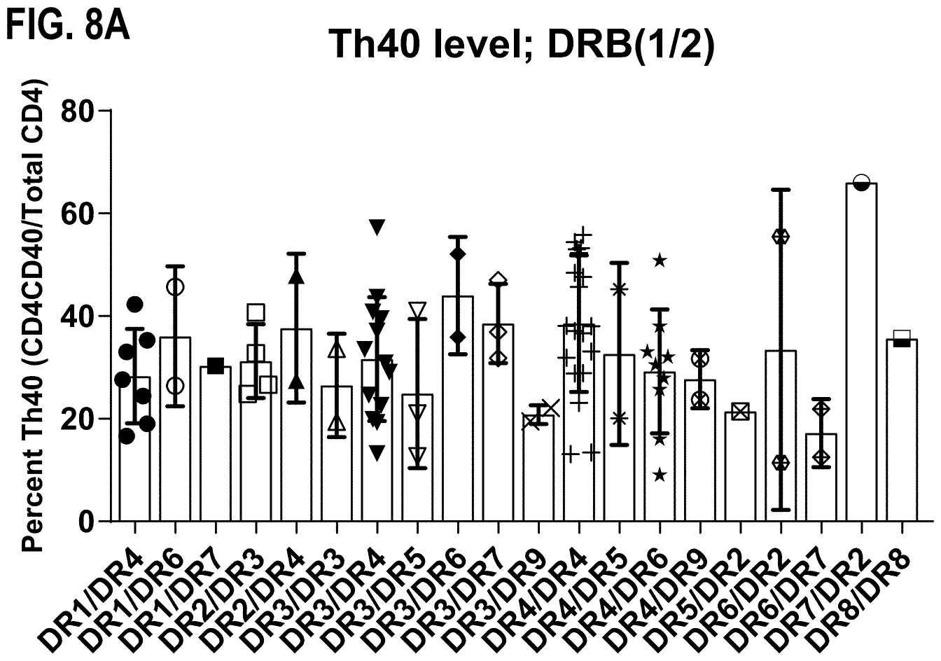

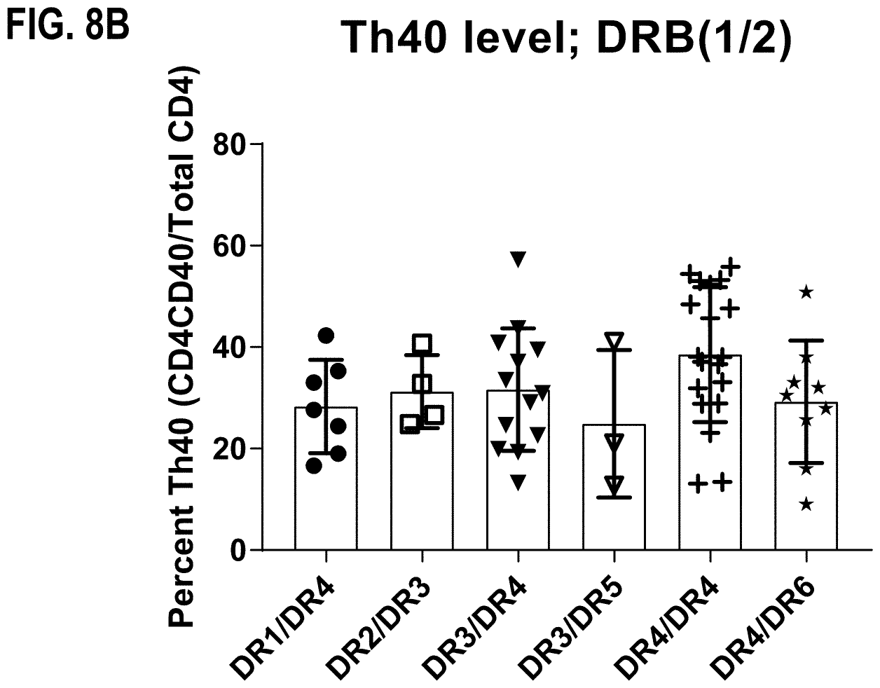

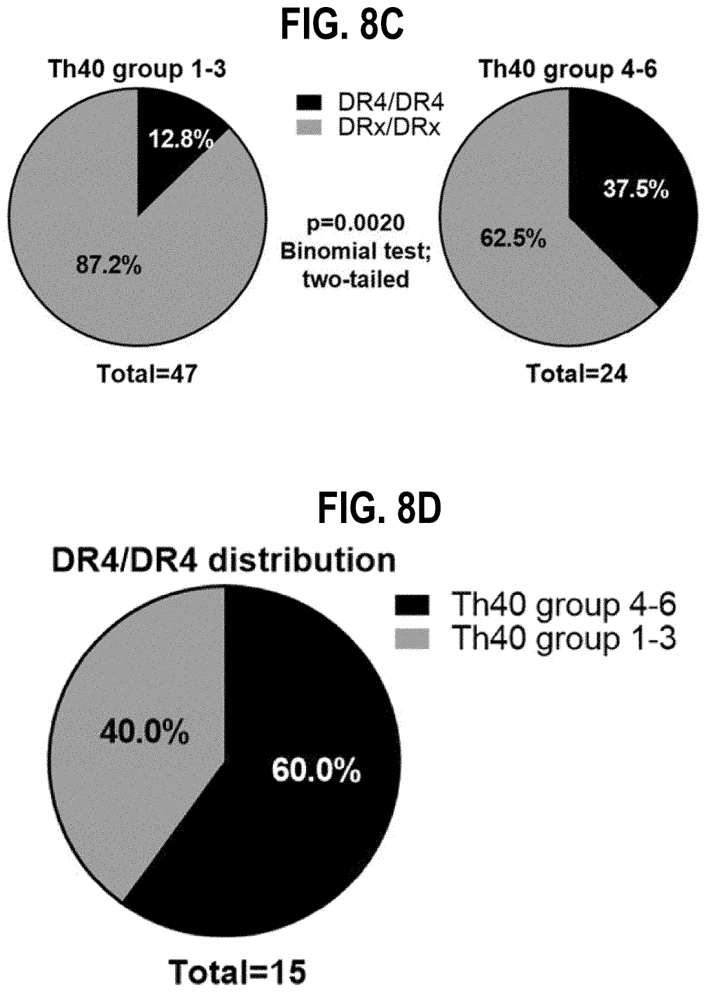

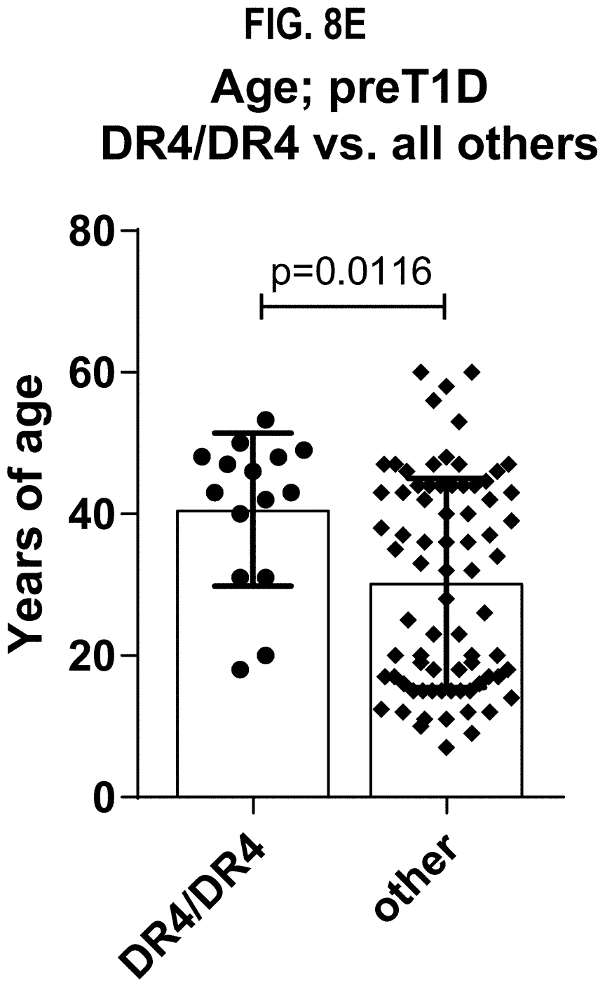

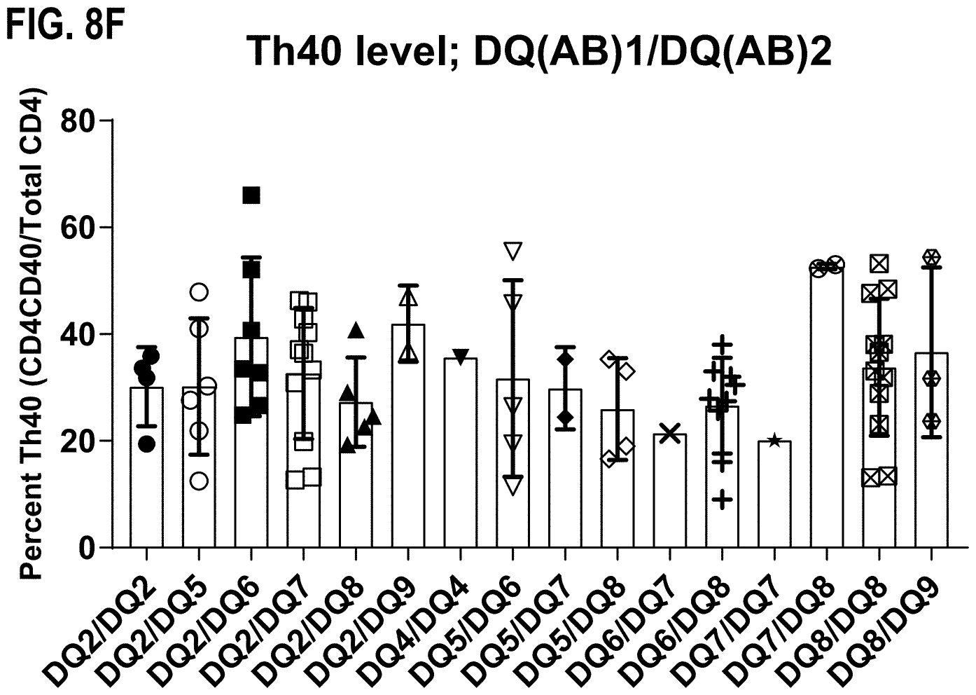

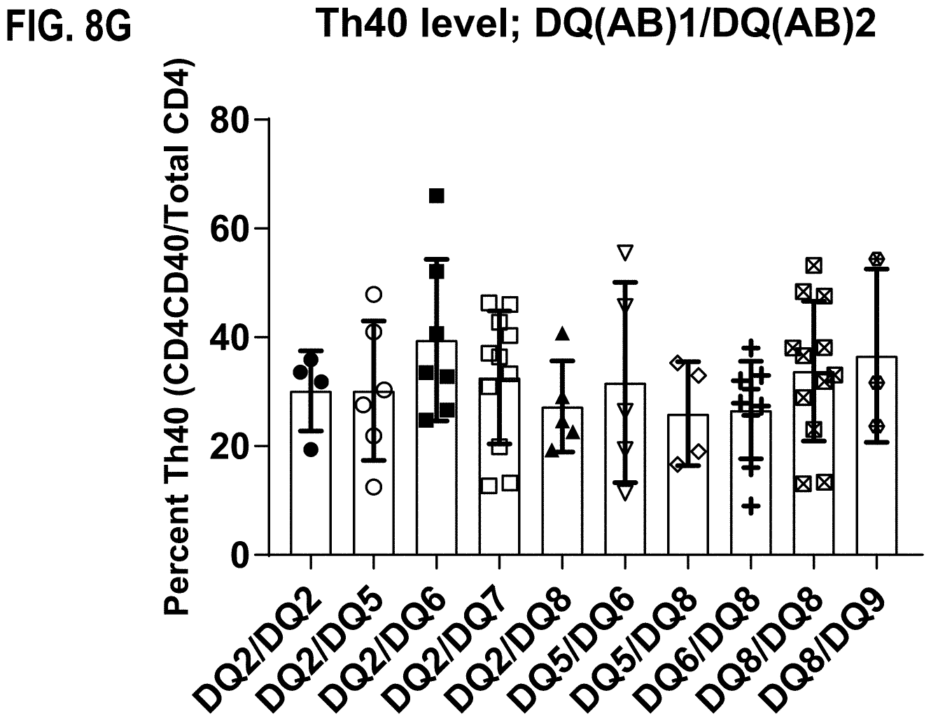

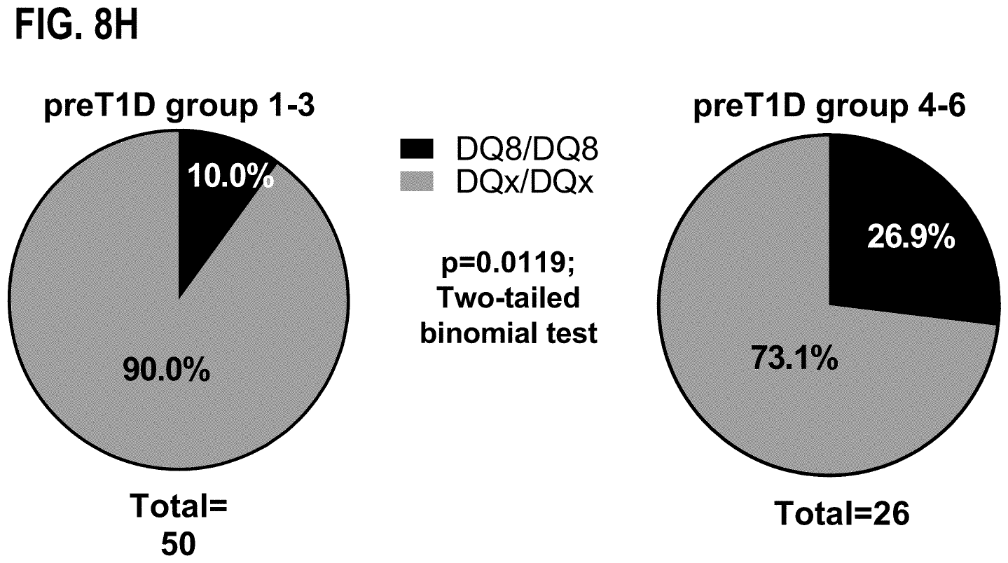

Previously we demonstrated that peripheral blood Th40 cell level in diagnosed long-term T1D subjects does not correlate with HLA haplotype (9). However, this was done on the general population, comparing control and T1D subjects. Here however, the subjects were recruited based on diabetes associated HLAs or being a first degree relative of a T1D subject. We performed HLA haplotype analysis on the preT1D samples. Plotting all subjects with known HLA-DRB haplotype, it was clear that there was a wide representation of haplotypes but with no significant difference in Th40 cell percentage between the haplotype groups (FIG. 8A). However, several haplotypes were represented by only one or two preT1D subjects. Therefore, we removed those from the analysis to better discern whether any differences were present among the remaining, better-represented groups (FIG. 8B). Again, no significant difference in Th40 cell level relative to HLA-DRB was found, but there was a trend towards a higher Th40 cell level in the DR4/DR4 group. Because of this, we divided the subjects depicted in FIG. 8A into Th40 cell level groups, based on the groupings in Table 2 (FIG. 9B), and tallied the representation of subjects with DR4/DR4 in those groups. We found that 37.5% of the subjects in preT1D groups 4-6 were DR4/DR4 while only 12.8% in the preT1D groups 1-3 had that haplotype (FIG. 8C; p=0.0020; two-tailed binomial test). When considering only subjects with the DR4/DR4 haplotype and graphing their representation in Th40 cell level groups 1-3 and 4-6 they were clearly more represented in groups 4-6 (FIG. 8D). Interestingly, when we plotted the age of the DR4/DR4 subjects against all other haplotype combinations, the DR4/DR4 subjects were significantly older than the other subjects (FIG. 8E; mean 40.6 versus mean 30.3 years, respectively; p=0.0116; two-tailed t-test).

PreT1D Subjects with HLA DQ8/DQ8 Haplotype Are More Likely to be Found in Th40 Cell Level Groups 4-6.

Like HLA DR3 and DR4, HLA DQ2 and DQ8 are strongly associated with T1D (22). We performed a similar analysis on HLA-DQ as we did for HLA-DRB. Again, there was a wide representation of haplotypes but no significant difference in Th40 cell level (FIG. 8F). When haplotypes represented by only one or two subjects were removed from the analysis, there was still no significant difference (FIG. 8G). However, when we divided the subjects depicted in FIG. 8F into Th40 cell level groups, based on the groupings in Table 2 (FIG. 9B), and tallied the representation of subjects with DQ8/DQ8 in those groups, DQ8/DQ8 was clearly more represented in the preT1D groups 4-6 than in groups 1-3 (FIG. 8H; p=0.0119; two-tailed binomial test).

FIG. 1A and FIG. 1B. Stratified PreT1D subjects parallel relationships found in between control and T1D subjects. FIG. 1A: PBMC from non-autoimmune control subjects and long standing T1D subjects were stained for Th40 cell levels, total CD4 and CD8 levels, and CD45RO levels in the CD4lo (Th40 cells) and CD4hi (conventional CD4 T cells) and run in flow cytometry. Cells were gated on FSC/SSC for live cells then gates were set based on isotype controls. FIG. 1B: PreT1D subjects were stratified into groups as shown in Table 2 (FIG. 9B) then subjects in groups 1-4 were compared to subjects in groups 5-6 by staining and gating as in A. Significant differences in both A and B were calculated by two-tailed t-test; p-values are noted in the figure and the manuscript text.

FIG. 2A and FIG. 2B provide graphs of conventional CD4 T cells from stratified pre-T1D subjects. Conventional CD4 T cells from stratified preT1D subjects demonstrate a difference in basal cytokine levels. PBMC were stained for CD4, CD3, and CD40 as well as for intracellular cytokines. Cells were gated on FSC/SSC for live cells then on CD4lo (Th40 cells) and CD4hi (conventional CD4 T cells) based on isotype control and CD3 expression was confirmed. PreT1D subjects were stratified into groups as shown in Table 2 (FIG. 9B) then subjects in groups 1-4 were compared to subjects in groups 5-6. Intracellular cytokine was measured by using the CD4⁻CD40⁻ cells in each sample as a built-in negative control. Percentages of IL-2, IL-4, IL-6, IL-10, IL-17, IFNγ, and TNFα content in CD4hi (left panel) and CD4lo (right panel) are shown. Significant differences were calculated by one-tailed t-test; p-values are noted in the figure and the manuscript text.

FIG. 3A-1, FIG. 3A-2, FIG. 3B, and FIG. 3C. Stratification of preT1D subjects reveal a different inducible cytokine profile in subjects with high Th40 cell levels. PreT1D subjects were stratified into groups as shown in Table 2 (FIG. 9B) then subjects in groups 1-4 were compared to subjects in groups 5-6. T cells were incubated with antigen loaded APC overnight then Brefeldin A was added. Cells were stained for CD4, CD3, and CD40 as well as for intracellular cytokines. Cells were gated on FSC/SSC for live cells then on CD4⁺CD40⁺ (Th40 cells) based on isotype control and CD3 expression was confirmed. Intracellular cytokines were measured by using isotype control and the CD4⁻CD40⁻ cells in each sample as a built in negative control. Percentages of IL-4, IL-10, IL-17, IFNγ, and TNFα content were graphed for each subject, creating a cytokine profile. (A) Representative cytokine profiles from subjects in groups 1-4 (top panels) and groups 5-6 (bottom panels). (B) Pie charts representing the subjects with high IL-4/IL-17 versus low IL-4/IL-17 profiles in preT1D groups 1-4 (left) and groups 5-6 (right). (C) Pie charts representing the subjects with high IL-4/IL-17 versus low IL-4/IL-17 profiles in preT1D groups 1-3 (left) and groups 4-6 (right). Significant differences in B and C were calculated by two-tailed binomial test; p-values are noted in the figure.

FIG. 4A, FIG. 4B, FIG. 4C, FIG. 4D, FIG. 4E-1, and FIG. 4E-2 provides dot plots, histograms, and charts of differences in presence of biomarkers. Stratified preT1D groups demonstrate differences in the presence of CD4/CD8 DP cells. PreT1D subjects were stratified into groups as shown in Table 2 (FIG. 9B) then subjects in groups 1-4 were compared to subjects in groups 5-6. PBMC were stained for CD4, CD3, CD8, and CD40. Cells were gated on FSC/SSC for live cells then CD4 and CD40 was plotted and gated based on isotype controls and CD3 expression was confirmed. (A) Representative CD4/CD40 dot plots from groups 1-4 (left) and groups 5-6 (right). (B) Histogram depicting CD8 content in the CD4 intermediate population (arrow from dot plot in A) from groups 5-6. (C) Pie charts representing the number of subjects with/without DP population in groups 1-4 (left) and groups 5-6 (right). (D) Pie charts representing the number of subjects with/without DP population in groups 1-3 (left) and groups 4-6 (right). (E) PBMC from two preT1D samples and two longstanding T1D samples were assayed in western blots for CD4 (left) and CD8 (right) after sorting on CD8 and CD4, respectively, was performed. Significant differences in C and D were calculated by two-tailed binomial test; p-values are noted in the figure.

FIG. 5A, FIG. 5B-1, and FIG. 5B-2 provide graphs of Th40 cell levels in subjects. Th40 cell levels in preT1D subjects do not correlate with age or antibody status. PBMC from preT1D subjects were stained for CD4, CD3, and CD40. Cells were gated on FSC/SSC for live cells then CD4/CD40 was plotted and gated based on isotype controls and CD3 expression confirmed. Th40 cell levels of PreT1D subjects were plotted according to (A) age groups or (B) number of autoantibodies (Ab; left panel—all subjects, right panel—mean Th40 level trend).

FIG. 6A and FIG. 6B provide graphs of Th40 levels in subjects. New onset T1D subjects have lower Th40 levels than long standing T1D subjects and preT1D subjects experiencing IGT have higher Th40 levels than control subjects. PBMC from new onset T1D, longstanding T1D, preT1D experiencing IGT, and control subjects were stained for CD4, CD3, and CD40. Cells were gated on FSC/SSC for live cells then CD4/CD40 was plotted and gated based on isotype controls and CD3 expression confirmed. (A) Th40 cell levels in new onset T1D compared to long standing T1D. (B) Th40 cell levels in preT1D subjects experiencing IGT compared to control subjects. Significant differences in A and B were calculated by two-tailed t-test; p-values are noted in the figure.

FIG. 7A, FIG. 7B and FIG. 7C provides graphs of different Th40 cell levels from subjects. Different Th40 cell level patterns emerge from longitudinal studies of preT1D subjects. PBMC from preT1D subjects were stained for CD4, CD3, and CD40. Cells were gated on FSC/SSC for live cells then CD4/CD40 was plotted and gated based on isotype controls and CD3 expression confirmed. Th40 cell levels over time were plotted for each patient that had been tested more than once. Autoantibody (Ab) status and any impairment in glucose tolerance (IGT) is noted for each time point.

FIG. 8A, FIG. 8B, FIG. 8C, FIG. 8D, FIG. 8E, FIG. 8F, FIG. 8G, and FIG. 8H provide graphs and charts related to haplotypes in subjects. HLA DR4/DR4 and DQ8/DQ8 are more prevalent in preT1D subjects with higher Th40 cell levels. Th40 cell levels in preT1D subjects were plotted according to HLA-DR haplotypes for (A) all subjects or (B) HLA-DR haplotypes represented by more than two subjects. (C) PreT1D subjects were stratified into groups as shown in Table 2 (FIG. 9B) then subjects in groups 1-3 (left) were compared to subjects in groups 4-6 (right) for the representation of HLA DR4/DR4 versus all other DR-haplotypes (DRx/DRx). (D) Distribution of DR4/DR4 subjects only among preT1D groups 1-3 and 4-6. (E) Graph depicting the age of DR4/DR4 subjects compared to all other haplotype subjects. (F) Th40 cell level according to HLA-DQ haplotype in all subjects or (G) HLA-DQ haplotypes represented by more than two subjects. (H) PreT1D subjects were stratified into groups as shown in Table 2 (FIG. 9B) then subjects in groups 1-3 (left) were compared to subjects in groups 4-6 (right) for the representation of HLA DQ8/DQ8 versus all other DQ-haplotypes (DQx/DQx). Significant differences in C and H were calculated by two-tailed binomial test and in E by two-tailed t-test; p-values are noted in the figure.

FIGS. 9A and 9B provide tables related to the subjects described in examples hereof. FIG. 9A: Table 1 and FIG. 9B: Table 2 provide data regarding the subjects of the Study S described above. Table 1 shows the blood samples acquired from preT1D subjects enrolled in the TrialNet study. From this data, FIG. 9B: Table 2 was created that shows the relative Th40 cell levels. Using the determined mean and standard deviation (SD) of the Th40 cell level from non-autoimmune control subjects, Th40 relative groups were generated by cumulatively adding the control SD to the control mean. The groups were: Group 1—any value lower than control mean, Group 2—control mean+up to 1 SD, Group 3—control mean+1-2 SD, Group 4—control mean+2-3 SD, Group 5—control mean+3-4 SD, and Group 6—control mean+>4 SD. Based on these groups the samples were ranked from control, T1D, and T2D subjects (Table 2—FIG. 9B). As expected, T1D subjects clearly, and almost exclusively, fell into groups 4, 5, and 6. Also as expected, non-autoimmune T2D subjects fell into groups 1 and 2, the same as the control subjects (Table 2—FIG. 9B). When these groupings were applied to preT1D subjects, 35% of the subjects fell into groups 4-6, with over half of those in groups 5 and 6. Therefore, assuming that preT1D subjects can be divided into those that will develop T1D and those who will not, and that those groups will be defined by higher versus lower peripheral blood Th40 cell levels, the relationship between the groups was studied and compared it to the relationship between control and T1D subjects.

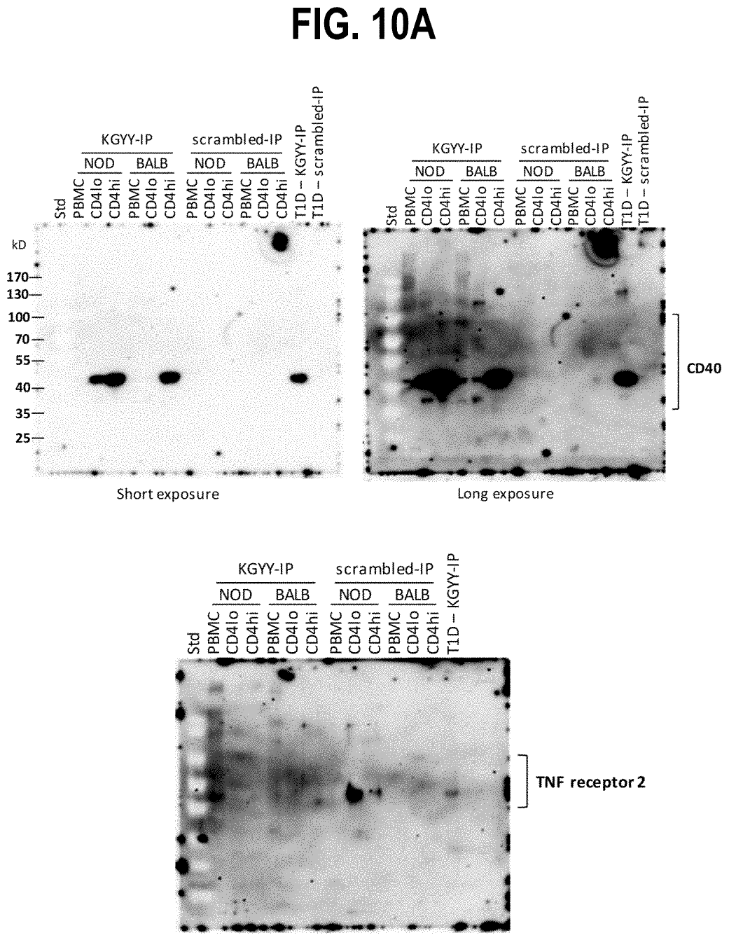

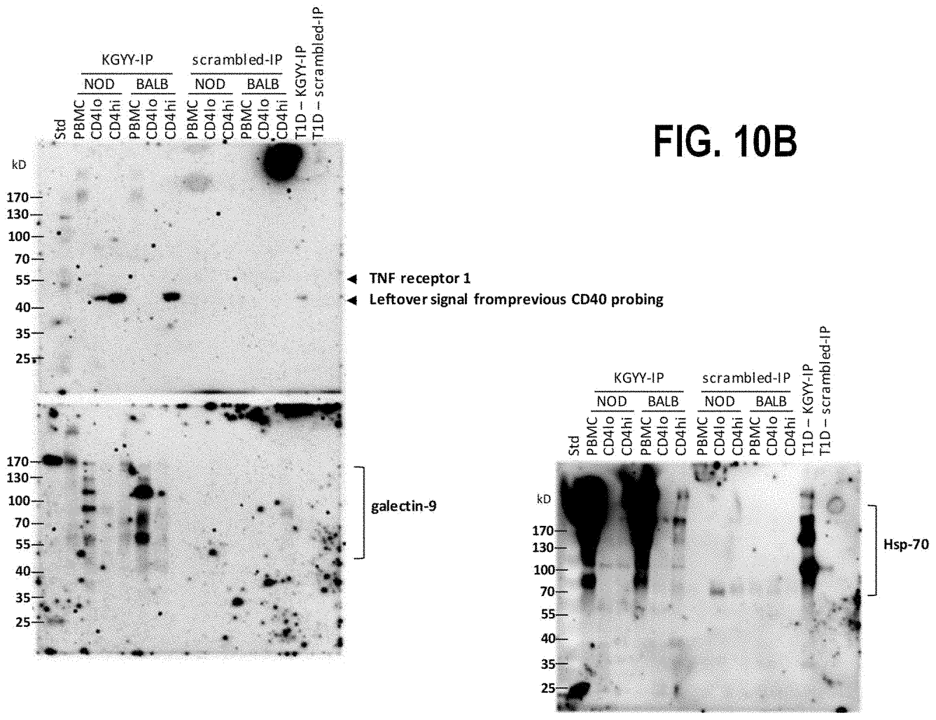

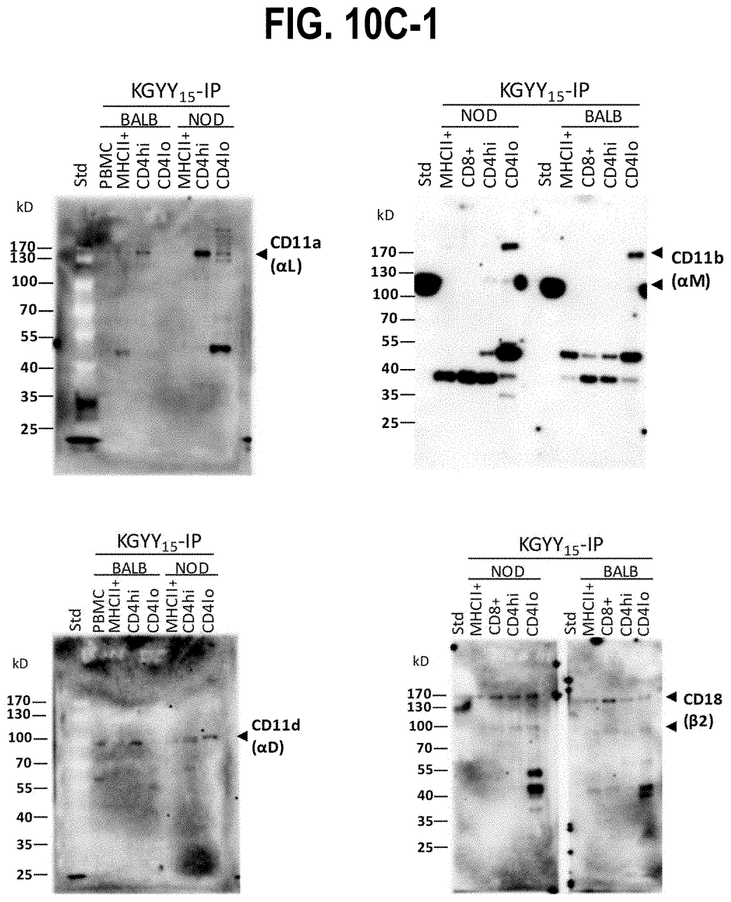

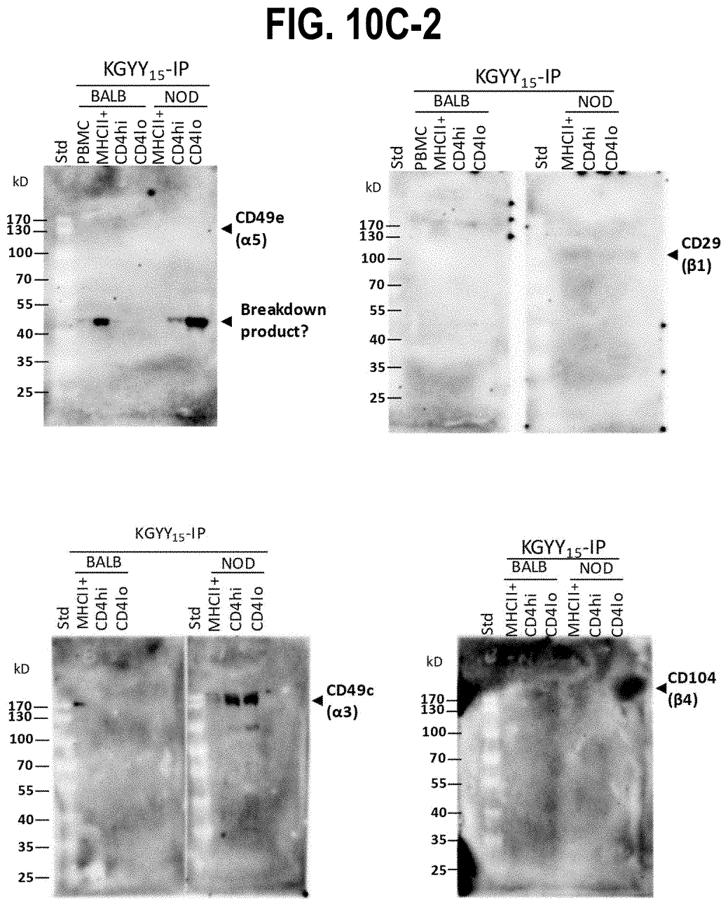

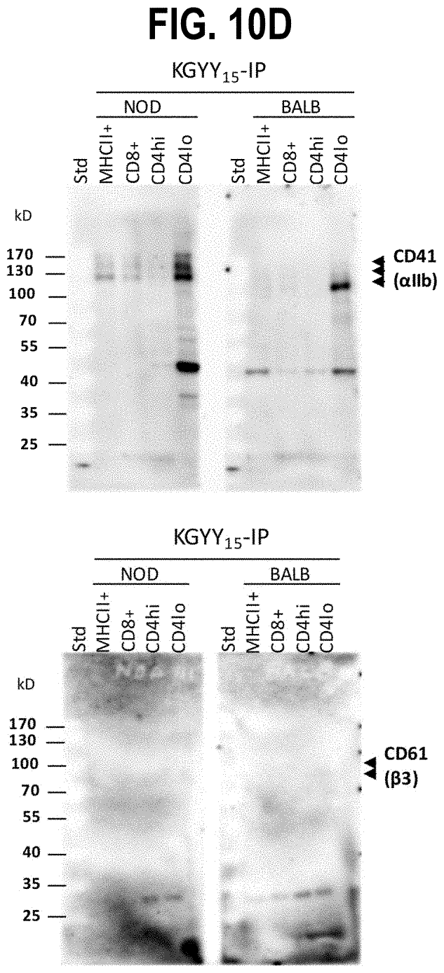

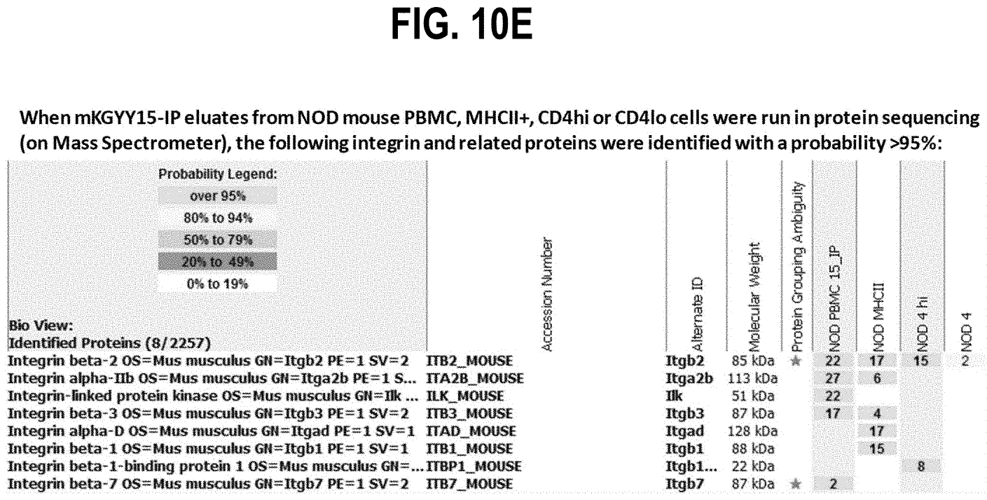

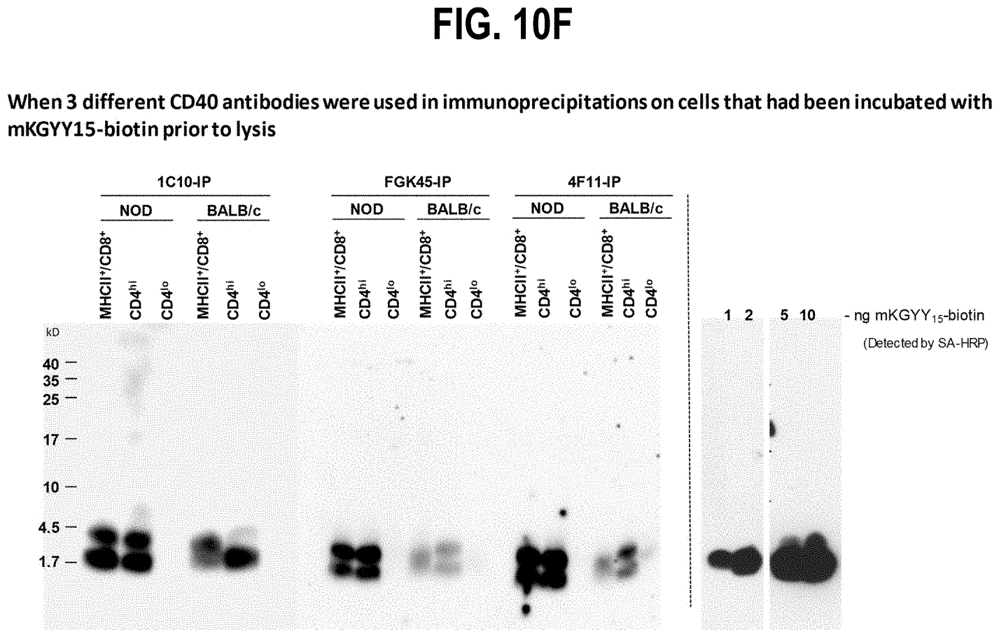

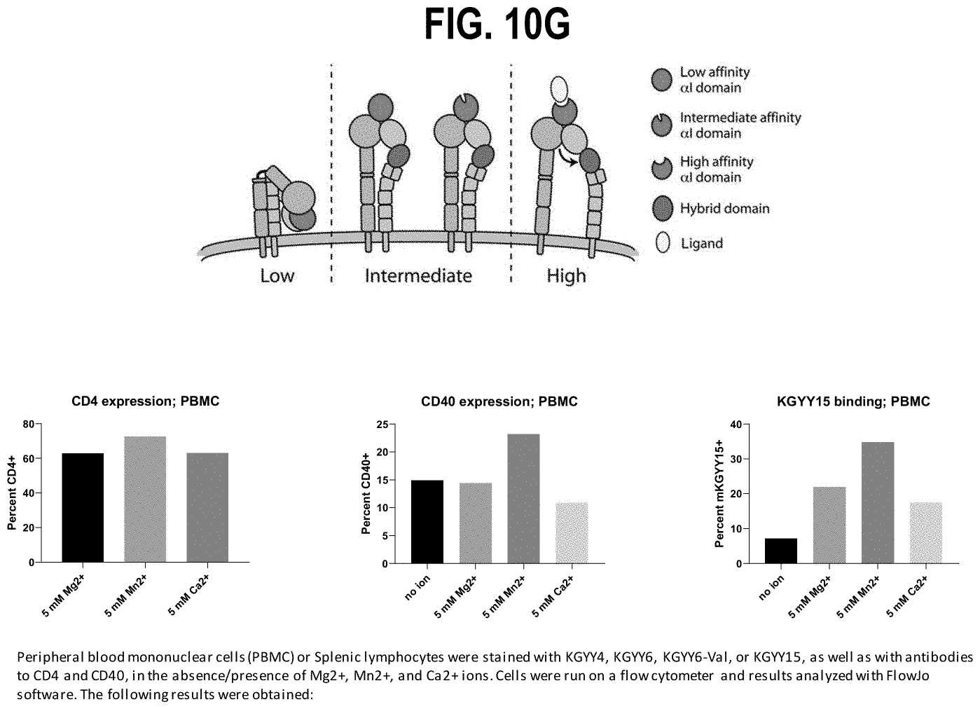

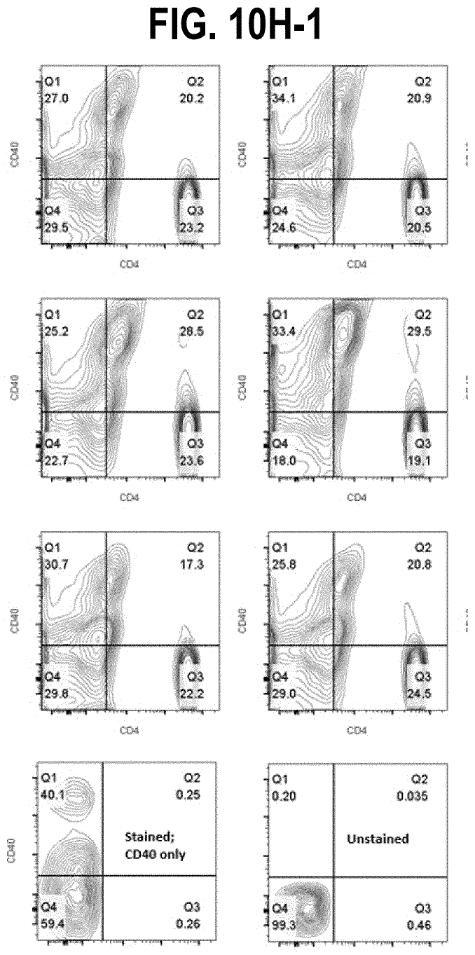

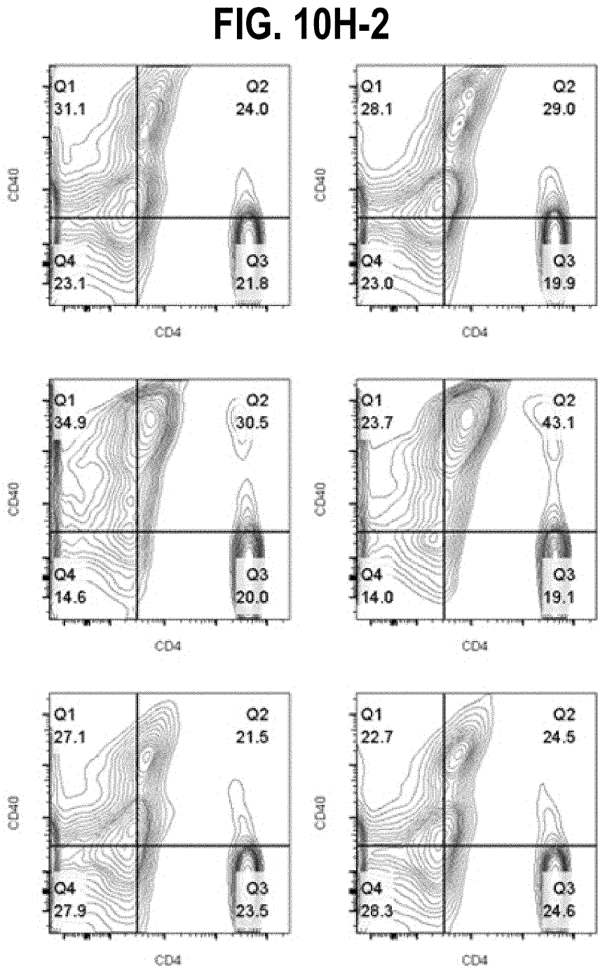

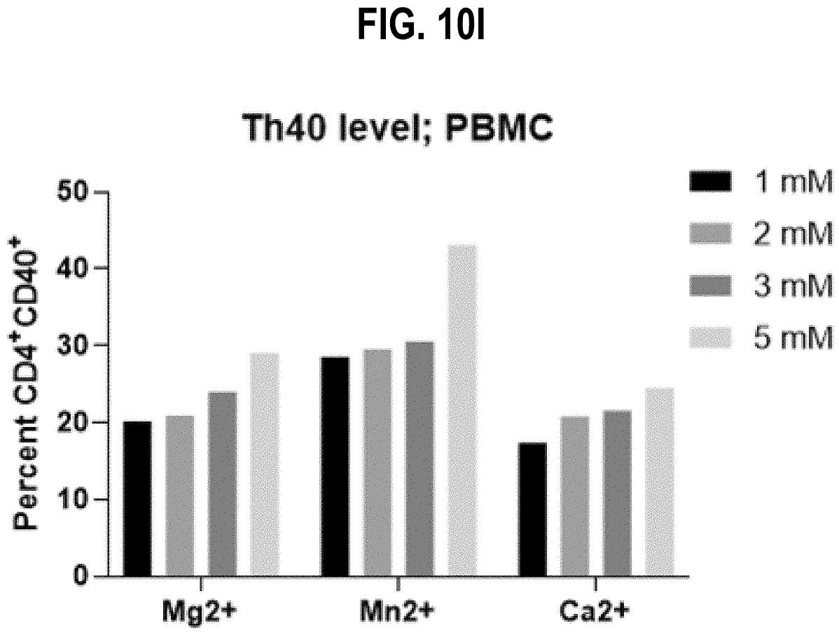

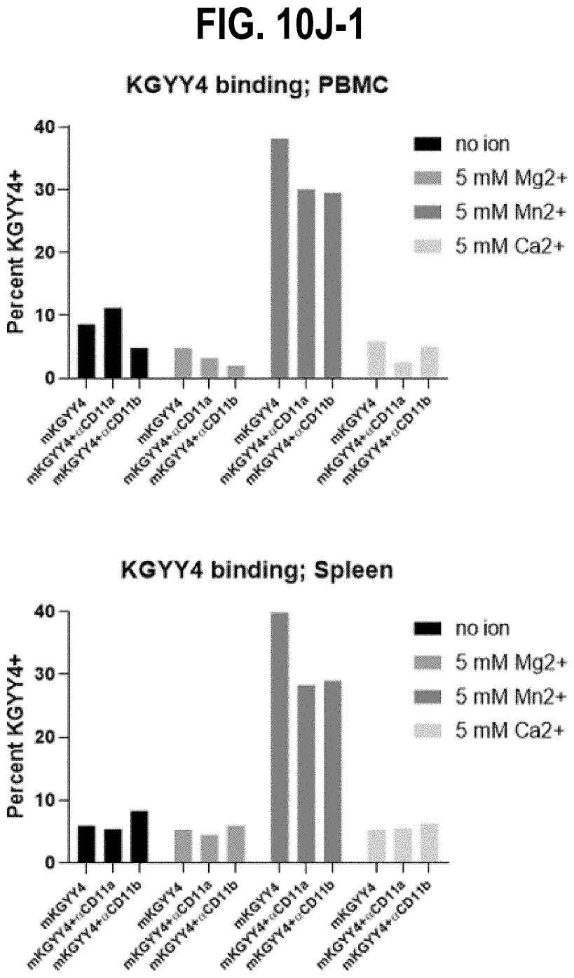

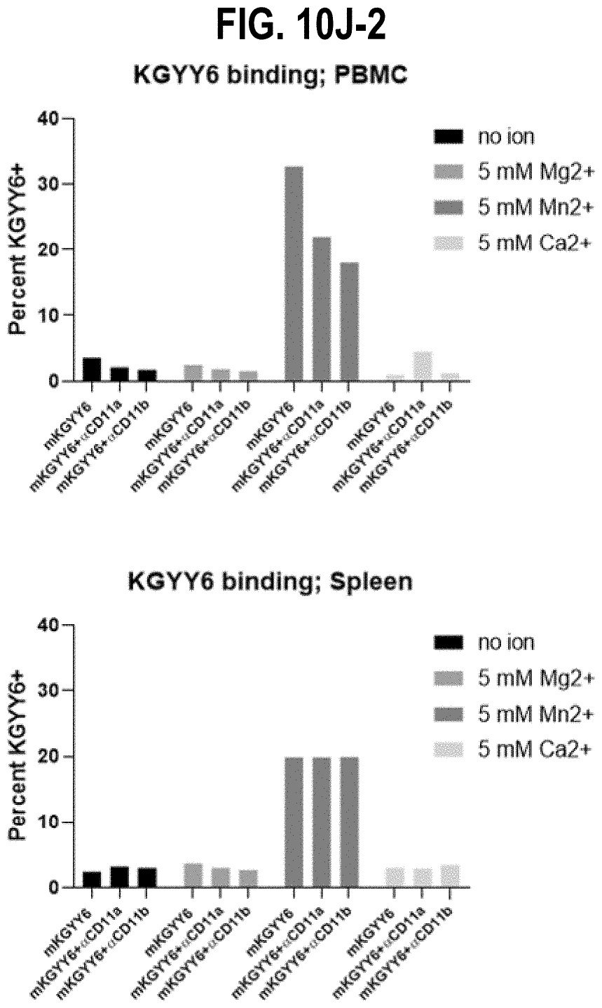

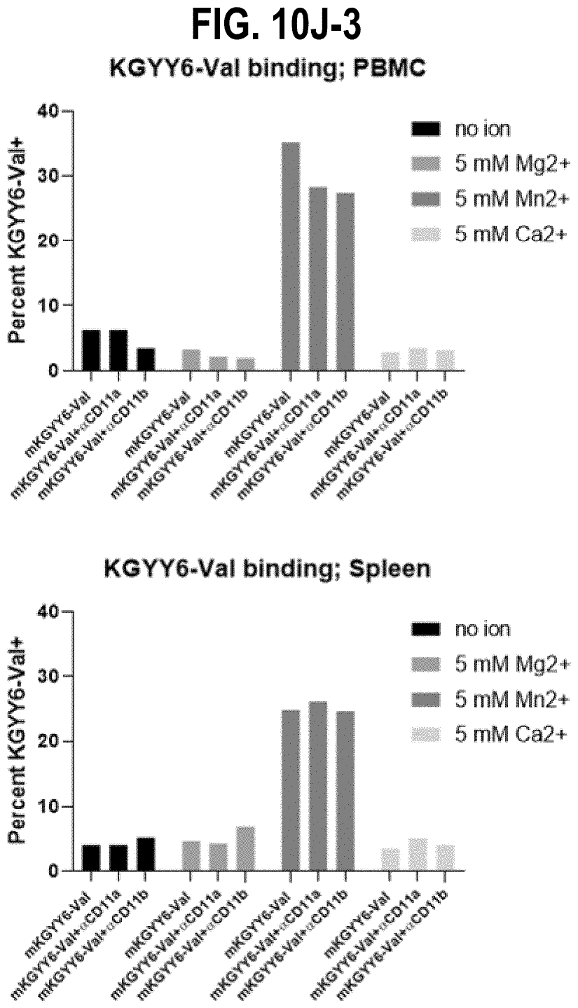

FIG. 10A, FIG. 10B, FIG. 10C-1, FIG. 10C-2, FIG. 10D, FIG. 10E, FIG. 10F, FIG. 10G, FIG. 10H-1, FIG. 10H-2, FIG. 10I, FIG. 10J-1, FIG. 10J-2, and FIG. 10J-3 provide blots, tables, graphs, charts, and plots related to additional biomarkers, receptors, and peptides that may bind thereto. The blots of FIG. 10A, FIG. 10B, FIGS. 10C-1, 10C-2 and FIG. 10D provide immunoprecipitation results of western blots. KGYY15 immunoprecipitated primarily αM, αD and β2 from CD4lo cells (CD4loCD40+; Th40), more prominently from autoimmune NOD cells than from control Balb/c cells. CD11a was also immunoprecipitated but only from NOD CD4lo cells and there were several differentially glycosylated (presumably) bands. Interestingly αIIb was immunoprecipitated but not β3, which forms the platelet integrin used in clot formation. KGYY15 immunoprecipitated primarily αL, αD and β2 from CD4hi cells (CD4+CD40−), αL more prominently from autoimmune NOD and αD from control Balb/c cells. FIG. 10E provides a table of when KGYY15 eluates from mouse PBMR, MHCII+, CD4hi or CD4lo cells in protein sequencing, for integrins and proteins listed in FIG. 10E. FIG. 10F provides results of a western blot when 3 different CD40 antibodies were used in immunoprecipitations on cells that had been incubated with mKGYY15-biotin prior to lysis. FIG. 10H-1 and FIG. 10H-2, FIG. 10I, and FIG. 10J-1, FIG. 10J-2, and FIG. 10J-3 provide additional binding tests and studies that were done regarding peptides of KGYY-4, KGYY-6, and KGYY6-Val.

### REFERENCES

1. Bodin J, Stene L C, Nygaard U C. Can exposure to environmental chemicals increase the risk of diabetes type 1 development? Biomed Res Int. 2015; 2015:208947.

2. Battaglia M, Anderson M S, Buckner J H, Geyer S M, Gottlieb P A, Kay T W H, Lernmark A, Muller S, Pugliese A, Roep B O, Greenbaum C J, Peakman M. Understanding and preventing type 1 diabetes through the unique working model of TrialNet. Diabetologia. 2017.

3. von Herrath M, Sanda S, Herold K. Type 1 diabetes as a relapsing-remitting disease? Nature reviews. 2007; 7(12):988-994.

4. Aly H, Gottlieb P. The honeymoon phase: intersection of metabolism and immunology. Curr Opin Endocrinol Diabetes Obes. 2009; 16(4):286-292.

5. Baker R L, Jamison B L, Wiles T A, Lindsay R S, Barbour G, Bradley B, Delong T, Friedman R S, Nakayama M, Haskins K. CD4 T Cells Reactive to Hybrid Insulin Peptides Are Indicators of Disease Activity in the NOD Mouse. Diabetes. 2018; 67(9):1836-1846.

6. Delong T, Wiles T A, Baker R L, Bradley B, Barbour G, Reisdorph R, Armstrong M, Powell R L, Reisdorph N, Kumar N, Elso C M, DeNicola M, Bottino R, Powers A C, Harlan D M, Kent S C, Mannering S I, Haskins K. Pathogenic CD4 T cells in type 1 diabetes recognize epitopes formed by peptide fusion. Science (New York, N.Y. 2016; 351(6274):711-714.

7. Wiles T A, Delong T, Baker R L, Bradley B, Barbour G, Powell R L, Reisdorph N, Haskins K. An insulin-IAPP hybrid peptide is an endogenous antigen for CD4 T cells in the non-obese diabetic mouse. Journal of autoimmunity. 2017; 78:11-18.

8. Wagner D H. Of the multiple mechanisms leading to type 1 diabetes, T cell receptor revision may play a prominent role (is type 1 diabetes more than a single disease?). Clinical and experimental immunology. 2016; 185(3):271-280.

9. Waid D M, Wagner R J, Putnam A, Vaitaitis G M, Pennock N D, Calverley D C, Gottlieb P, Wagner D H, Jr. A unique T cell subset described as CD4loCD40+ T cells (TCD40) in human type 1 diabetes. Clinical immunology (Orlando, Fla. 2007; 124(2):138-148.

10. Waid D M, Vaitaitis G M, Wagner D H, Jr. Peripheral CD4loCD40+ auto-aggressive T cell expansion during insulin-dependent diabetes mellitus. European journal of immunology. 2004; 34(5):1488-1497.

11. Vaitaitis G M, Wagner D H, Jr. High distribution of CD40 and TRAF2 in Th40 T cell rafts leads to preferential survival of this auto-aggressive population in autoimmunity. PLoS One. 2008; 3(4):e2076.

12. Waid D M, Vaitaitis G M, Pennock N D, Wagner D H, Jr. Disruption of the homeostatic balance between autoaggressive (CD4+CD40+) and regulatory (CD4+CD25+FoxP3+) T cells promotes diabetes. J Leukoc Biol. 2008; 84(2):431-439.

13. Vaitaitis G M, Waid D M, Wagner D H, Jr. The expanding role of TNF-Receptor Super Family member CD40 (tnfrsf5) in Autoimmune Disease: Focus on Th40 cells. Current Immunology Reviews. 2010; Volume 6 (Number 2):130-136.

14. Wagner D H, Jr., Vaitaitis G, Sanderson R, Poulin M, Dobbs C, Haskins K. Expression of CD40 identifies a unique pathogenic T cell population in type 1 diabetes. Proc Natl Acad Sci USA. 2002; 99(6):3782-3787.

15. Vaitaitis G M, Waid D M, Yussman M G, Wagner D H, Jr. CD40-mediated signalling influences trafficking, T-cell receptor expression, and T-cell pathogenesis, in the NOD model of type 1 diabetes. Immunology. 2017; 152(2):243-254.

16. Vaitaitis G M, Poulin M, Sanderson R J, Haskins K, Wagner D H, Jr. Cutting edge: CD40-induced expression of recombination activating gene (RAG) 1 and RAG2: a mechanism for the generation of autoaggressive T cells in the periphery. J Immunol. 2003; 170(7):3455-3459.

17. Vaitaitis G M, Wagner D H, Jr. CD40 interacts directly with RAG1 and RAG2 in autoaggressive T cells and Fas prevents CD40-induced RAG expression. Cellular & molecular immunology. 2013; 10(6):483-489.

18. Vaitaitis G M, Olmstead M H, Waid D M, Carter J R, Wagner D H, Jr. A CD40-targeted peptide controls and reverses type 1 diabetes in NOD mice. Diabetologia. 2014; 57(11):2366-2373.

19. Hoxie J A, Matthews D M, Callahan K J, Cassel D L, Cooper R A. Transient modulation and internalization of T4 antigen induced by phorbol esters. J Immunol. 1986; 137(4):1194-1201.

20. Ehlers M R, Rigby M R. Targeting memory T cells in type 1 diabetes. Curr Diab Rep. 2015; 15(11):84.

21. Vehik K, Beam C A, Mahon J L, Schatz D A, Haller M J, Sosenko J M, Skyler J S, Krischer J P, TrialNet Natural History Study G. Development of autoantibodies in the TrialNet Natural History Study. Diabetes care. 2011; 34(9):1897-1901.

22. van Lummel M, van Veelen P A, de Ru A H, Pool J, Nikolic T, Laban S, Joosten A, Drijfhout J W, Gomez-Tourino I, Arif S, Aanstoot H J, Peakman M, Roep B O. Discovery of a Selective Islet Peptidome Presented by the Highest-Risk HLA-DQ8trans Molecule. Diabetes. 2016; 65(3):732-741.

23. Walker L S, von Herrath M. CD4 T cell differentiation in type 1 diabetes. Clinical and experimental immunology. 2016; 183(1):16-29.

24. Nascimbeni M, Shin E C, Chiriboga L, Kleiner D E, Rehermann B. Peripheral CD4(+)CD8(+) T cells are differentiated effector memory cells with antiviral functions. Blood. 2004; 104(2):478-486.

25. Quandt D, Rothe K, Scholz R, Baerwald C W, Wagner U. Peripheral CD4CD8 double positive T cells with a distinct helper cytokine profile are increased in rheumatoid arthritis. PLoS One. 2014; 9(3):e93293.

26. Kishimoto H, Sprent J. A defect in central tolerance in NOD mice. Nature immunology. 2001; 2(11):1025-1031.

27. Vaitaitis G M, Wagner D H, Jr. CD40 glycoforms and TNF-receptors 1 and 2 in the formation of CD40 receptor(s) in autoimmunity. Mol Immunol. 2010; 47(14):2303-2313.

28. Thomas N J, Jones S E, Weedon M N, Shields B M, Oram R A, Hattersley A T. Frequency and phenotype of type 1 diabetes in the first six decades of life: a cross-sectional, genetically stratified survival analysis from UK Biobank. Lancet Diabetes Endocrinol. 2018; 6(2):122-129.

29. Waid D M, Schreiner T, Vaitaitis G, Carter J R, Corboy J R, Wagner D H, Jr. Defining a new biomarker for the autoimmune component of Multiple Sclerosis: Th40 cells. Journal of neuroimmunology. 2014; 270(1-2):75-85.

1. (canceled)

2. (canceled)

3. (canceled)

4. (canceled)

5. (canceled)

6. (canceled)

7. (canceled)

8. (canceled)

9. (canceled)

10. A method for diagnosing a subject as having type 1 diabetes, comprising:
obtaining a blood sample from a subject;
separating the blood sample in to at least two sub-samples;
determining a percentage of cells expressing Th40;
determining a percentage of cells expressing one or more markers selected from one or more of CD4, CD8, CD40, CD25, CD45, TCRV8.3+, CXCR3 and CCR5;
comparing the percentage of Th40 cells to one or both of a control sample or a standard value;
comparing the percentage of one or more of CD4, CD8, CD40, CD25, CD45, TCRV8.3+, CXCR3 and CCR5 cells to one or both of a control sample or a standard value;
and diagnosing type 1 diabetes in the subject as an increase in the percentage of Th40 and an increase or decrease in the percentage of one or more of CD4, CD8, CD40, CD25, CD45, TCRV8.3+, CXCR3 and CCR5 cells in the sample from the subject relative to the one or both of a control sample or standard being indicative of type 1 diabetes in the subject.

11. A method for diagnosing type 1 diabetes in a subject comprising:
obtaining a blood sample from a subject;
separating the blood sample in to at least two sub-samples;
determining a percentage of cells expressing Th40;
determining a percentage of cells expressing a biomarker from the group comprising one or more of CD11a, CD11b, CD11d, CD18, CD29, CD41, CD49c, CD49e, CD61, and CD104 from the subject;
comparing the percentage of cells expressing Th40 to one or both of a control sample or standard value;
comparing the percentage of one or more of CD11a, CD11b, CD11d, CD18, CD29, CD41, CD49c, CD49e, CD61, and CD104 cells to one or both of a control sample or a standard value; and
diagnosing type 1 diabetes in the subject, an increase or decrease in the percentage of Th40 cells and one or more of CD11 a, CD11b, CD11d, CD18, CD29, CD41, CD49c, CD49e, CD61, and CD104 cells in the sample from the subject relative to the one or more of a control sample or standard is indicative of type 1 diabetes in the subject.

12. (canceled)

13. The method of claim 10, the control sample being a sample from at least one subject known not to have type 1 diabetes.

14. The method of claim 10, the control sample being a sample from at least one subject known to have type 1 diabetes.

15. The method of claim 10, the control sample being obtained from the subject at an earlier date.

16. The method of claim 10, the sample being one or more of whole blood, plasma, serum, or a subfraction of whole blood.

17. The method of claim 10, the method further comprising staining with a labeled antibody that specifically recognizes a protein selected from one or more of CD4, CD8, CD40, CD25, CD45, TCRV8.3+, CXCR3, CCR5, CD11a, CD11b, CD11d, CD18, CD29, CD41, CD49c, CD49e, CD61, and CD104 and analyzing the stained cells by flow cytometry to determine the percentage of stained cells in the sample.

18. The method of claim 10, the subject being selected from a human, a non-human primate, rodents, dogs, cats, and/or horses.

19. A method for diagnosing type 1 diabetes in a subject comprising:
obtaining a blood sample from a subject;
determining the HLA haplotype of the subject;
determining a percentage of cells expressing Th40;
determining whether one or more autoantibodies are produced by the subject;
diagnosing type 1 diabetes in the subject, where an increase or decrease in the percentage of Th40 cells and one or more of the DR4/DR4 or DQ8/DQ8 haplotype as well as two or more autoantibodies in the sample from the subject relative to the one or more of a control sample or standard is indicative of type 1 diabetes in the subject.

20. (canceled)

21. (canceled)

22. The method of claim 11, the control sample being a sample from at least one subject known not to have type 1 diabetes.

23. The method of claim 11, the control sample being a sample from at least one subject known to have type 1 diabetes.

24. The method of claim 11, the control sample being a sample obtained from the subject at an earlier date.

25. The method of claim 11, the sample being one or more of whole blood, plasma, serum, or a subfraction of whole blood.

26. The method of claim 11, the method further comprising staining with a labeled antibody that specifically recognizes a protein selected from one or more of CD4, CD8, CD40, CD25, CD45, TCRV8.3+, CXCR3, CCR5, CD11a, CD11b, CD11d, CD18, CD29, CD41, CD49c, CD49e, CD61, and CD104 and analyzing the stained cells by flow cytometry to determine the percentage of stained cells in the sample.

27. The method of claim 11, the subject being selected from a human, a non-human primate, rodents, dogs, cats, and/or horses.

28. The method of claim 19, the blood sample being one or more of whole blood, plasma, serum, or a subfraction of whole blood.

29. The method of claim 19, the control sample being a sample from at least one subject known not to have type 1 diabetes.

30. The method of claim 19, the control sample being a sample from at least one subject known to have type 1 diabetes.

31. The method of claim 19, the subject being selected from a human, a non-human primate, rodents, dogs, cats, and/or horses.

32. The method of claim 19, further comprising measuring one or more of CD4, CD8, CD40, CD25, CD45, TCRV8.3+, CXCR3, CCR5, CD11a, CD11b, CD11d, CD18, CD29, CD41, CD49c, CD49e, CD61, CD104, IL-2, IL-4, IL-6, IL-10, IL-17, IFNγ, and/or TNFα.
